(19) United States
(12) Patent Application Publication
(10) Pub. No.: US 2022/0098201 A1
(43) Pub. Date: Mar. 31, 2022

(54) THE MONOHYDRATE OF ROGARATINIB HYDROCHLORIDE AND SOLID STATES THEREOF

(71) Applicants: Bayer Aktiengesellschaft, Leverkusen (DE); Bayer Pharma Aktiengesellschaft, Berlin (DE)

(72) Inventors: Jörg GRIES, Haan (DE); Johannes PLATZEK, Berlin (DE); Claus-Christian HÄSELHOFF, Gladbeck (DE); Kai LOVIS, Dússeldorf (DE)

(73) Assignees: Bayer Aktiengesellschaft, Leverkusen (DE); Bayer Pharma Aktiengesellschaft, Berlin (DE)

(21) Appl. No.: 17/310,347

(22) PCT Filed: Jan. 27, 2020
(86) PCT No.: PCT/EP2020/051884

§ 371 Date: Jul. 28, 2021

(30) Foreign Application Priority Data
Jan. 31, 2019 (EP) 19154781.9

(51) Int. Cl. C07D 487/04

(52) U.S. Cl. CPC C07D 487/04; C07B 2200/13

(57) ABSTRACT

Compound (III) which is the crystalline form of [4-{[4-amino-6-(methoxymethyl)-5-(7-methoxy-5-methyl-1-benzothiophen-2-yl)pyrrolo[2, -f] [1,2,4]triazin-7-yl]methyl}piperazin-2-one hydrochloride] which is the monohydrate, processes for its preparation, pharmaceutical compositions comprising it and its use in the control of disorders, including cancer.

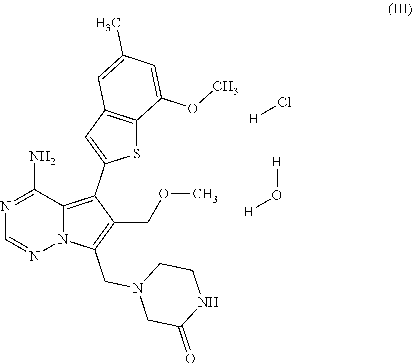 [SMILES: COCc1c(-c2cc3cc(C)cc(OC)c3s2)c2c(N)ncnn2c1CN1CCNC(=O)C1 ; O ; Cl]

### BACKGROUND OF THE INVENTION

There are many ways how cancers can arise which is one of the reasons why their therapy is difficult. One way that transformation of cells can occur is following a genetic alteration. The completion of the human genome project showed genomic instability and heterogeneity of human cancer genes. Recent strategies to identify these genetic alterations sped up the process of cancer-gene discovery. Gene abnormality can, for instance, lead to the overexpression of proteins, and hence to a non-physiological activation of these proteins. One family of proteins from which a number of onco-proteins derive are tyrosine kinases and in particular receptor tyrosine kinases (RTKs). In the past two decades, numerous avenues of research have demonstrated the importance of RTK-mediated signaling in adverse cell growth leading to cancer. In recent years, promising results have been achieved in the clinic with selective small-molecule inhibitors of tyrosine kinases as a new class of anti-tumorigenic agents [Swinney and Anthony, Nature Rev. Drug Disc. 10 (7), 507-519 (2011)].

Fibroblast growth factors (FGFs) and their receptors (FGFRs) form part of a unique and diverse signaling system which plays a key role in a variety of biological processes which encompass various aspects of embryonic development and adult pathophysiology [Itoh and Ornitz, J. Biochem. 149 (2), 121-130 (2011)]. In a spatio-temporal manner, FGFs stimulate through FGFR binding a wide range of cellular functions including migration, proliferation, differentiation, and survival.

The FGF family comprises 18 secreted polypeptidic growth factors that bind to four highly conserved receptor tyrosine kinases (FGFR-1 to -4) expressed at the cell surface. In addition, FGFR-5 can bind to FGFs but does not have a kinase domain, and therefore is devoid of intracellular signaling. The specificity of the ligand/receptor interaction is enhanced by a number of transcriptional and translational processes which give rise to multiple isoforms by alternative transcriptional initiation, alternative splicing, and C-terminal truncations. Various heparan sulfate proteoglycans (e.g. syndecans) can be part of the FGF/FGFR complex and strongly influence the ability of FGFs to induce signaling responses [Polanska et al., Developmental Dynamics 238 (2), 277-293 (2009)]. FGFRs are cell surface receptors consisting of three extracellular immunoglobulin-like domains, a single-pass transmembrane domain, and an intracellular dimerized tyrosine kinase domain. Binding of FGF bring the intracellular kinases into close proximity, enabling them to transphosphorylate each other. Seven phosphorylation sites have been identified (e.g., in FGFR-1 Tyr463, Tyr583, Tyr585, Tyr653, Tyr654, Tyr730, and Tyr766).

Some of these phosphotyrosine groups act as docking sites for downstream signalling molecules which themselves may also be directly phosphorylated by FGFR, leading to the activation of multiple signal transduction pathways. Thus, the MAPK signaling cascade is implicated in cell growth and differentiation, the PI3K/Akt signaling cascade is involved in cell survival and cell fate determination, while the PI3K and PKC signaling cascades have a function in the control of cell polarity. Several feedback inhibitors of FGF signaling have now been identified and include members of the Spry (Sprouty) and Sef (similar expression to FGF) families. Additionally, in certain conditions, FGFR is released from pre-Golgi membranes into the cytosol. The receptor and its ligand, FGF-2, are co-transported into the nucleus by a mechanism that involves importin, and are engaged in the CREB-binding protein (CBP) complex, a common and essential transcriptional co-activator that acts as a gene activation gating factor. Multiple correlations between the immunohistochemical expression of FGF-2, FGFR-1 and FGFR-2 and their cytoplasmic and nuclear tumor cell localizations have been observed. For instance, in lung adenocarcinomas this association is also found at the nuclear level, emphasizing an active role of the complex at the nucleus [Korc and Friesel, Curr. Cancer Drugs Targets 5, 639-651 (2009)].

FGFs are widely expressed in both developing and adult tissues and play important roles in a variety of normal and pathological processes, including tissue development, tissue regeneration, angio-genesis, neoplastic transformation, cell migration, cellular differentiation, and cell survival. Additionally, FGFs as pro-angiogenic factors have also been implicated in the emerging phenomenon of resistance to vascular endothelial growth factor receptor-2 (VEGFR-2) inhibition [Bergers and Hanahan, Nat. Rev. Cancer 8, 592-603 (2008)].

Recent oncogenomic profiles of signaling networks demonstrated an important role for aberrant FGF signaling in the emergence of some common human cancers [Wesche et al., Biochem. J. 437 (2), 199-213 (2011)]. Ligand-independent FGFR constitutive signaling has been described in many human cancers, such as brain cancer, head and neck cancer, gastric cancer and ovarian cancer. FGFR-mutated forms as well as FGFR-intragenic translocations have been identified in malignancies such as myeloproliferative diseases. Interestingly, the same mutations discovered to be the cause of many developmental disorders are also found in tumor cells (e.g., the mutations found in achondroplasia and thanatophoric dysplasia, which cause dimerization and thus constitutive activation of FGFR-3, are also frequently found in bladder cancer). A mutation that promotes dimerization is just one mechanism that can increase ligand-independent signaling from FGFRs. Other mutations located inside or outside of the kinase domain of FGFRs can change the conformation of the domain giving rise to permanently active kinases.

Amplification of the chromosomal region 8p11-12, the genomic location of FGFR-1, is a common focal amplification in breast cancer and occurs in approximately 10% of breast cancers, predominantly in oestrogen receptor-positive cancers. FGFR-1 amplifications have also been reported in non-small cell lung squamous carcinoma and are found at a low incidence in ovarian cancer, bladder cancer and rhabdomyosarcoma. Similarly, approximately 10% of gastric cancers show FGFR-2 amplification, which is associated with poor prognosis, diffuse-type cancers. Moreover, multiple single nucleotide polymorphisms (SNPs) located in FGFR-1 to -4 were found to correlate with an increased risk of developing selective cancers, or were reported to be associated with poor prognosis (e.g., FGFR-4 G388R allele in breast cancer, colon cancer and lung adenocarcinoma). The direct role of these SNPs to promote cancer is still controversial.

Potent FGFR inhibitors of general formula (I) were identified in WO 2013/087578, published 20 Jun. 2013:

### 6,7-disubstituted 5-(1-benzothiophen-2-yl)pyrrolo[2,1-f][1,2,4]triazin-4-amine Derivatives of the General Formula (A)

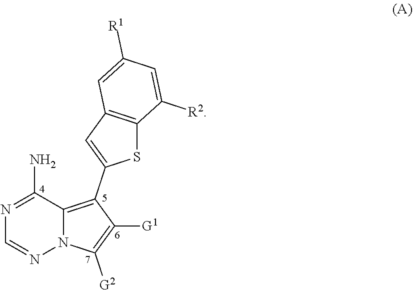

More particularly, a compound of the formula (I)

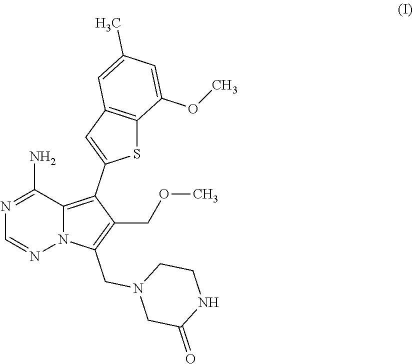 [SMILES: COCc1c(-c2cc3cc(C)cc(OC)c3s2)c2c(N)ncnn2c1CN1CCNC(=O)C1]

### 4-{[4-Amino-6-(methoxymethyl)-5-(7-methoxy-5-methyl-1-benzothiophen-2-yl)pyrrolo[2,1 f]¬[1,2,4]¬triazin-7-yl]methyl}piperazin-2-one

or a pharmaceutically acceptable salt, hydrate, or solvate thereof, which serves for production of medicaments and for production of medicaments for treatment and/or prophylaxis of proliferative disorders, such as cancer and tumor diseases, is a particularly potent FGFR inhibitor.

### 4-{[4-Amino-6-(methoxymethyl)-5-(7-methoxy-5-methyl-1-benzothiophen-2-yl)pyrrolo[2,1 f]¬[1,2,4]¬triazin-7-yl]methyl}piperazin-2-one has been Given the INN ROGARATINIB

Rogaratinib has valuable pharmacological properties and can be used for the prevention and treatment of disorders in humans and other mammals.

Rogaratinib is a potent inhibitor of the activity or expression of receptor tyrosine kinases, particularly of the FGFR kinases, and most notably of the FGFR-1 and FGFR-3 kinases. In certain embodiments, the disorders relating to the activity of FGFR kinases are proliferative disorders, in particular cancer and tumor diseases.

Synthesis of (I) has been described in WO 2013/087578 by two routes, which are illustrated in the following schemes. A synthetic route from WO 2013/087578 is described in Scheme 1:

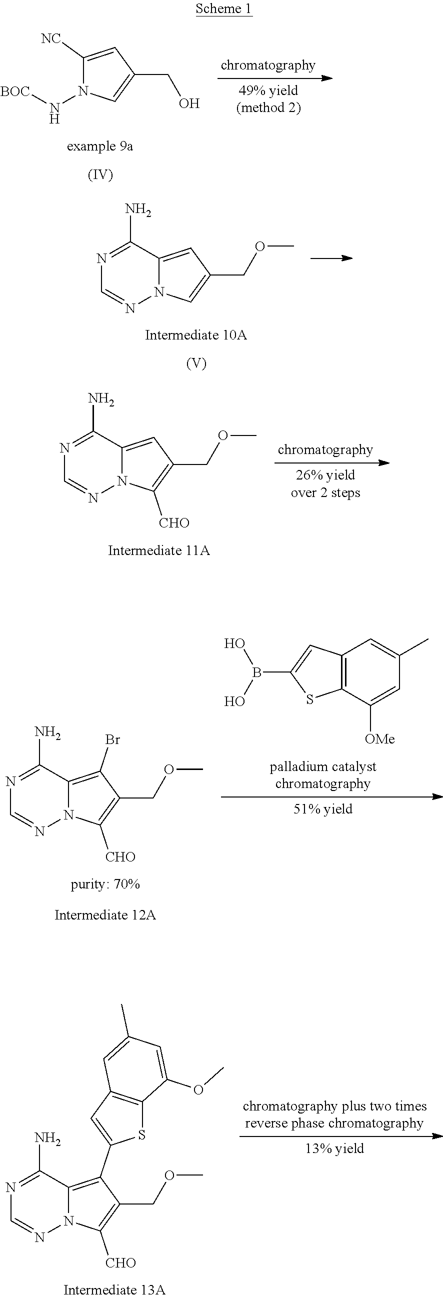

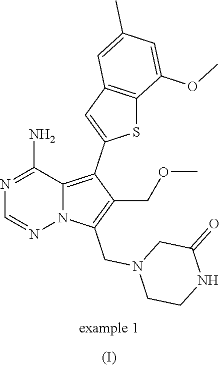 [SMILES: COCc1c(-c2cc3cc(C)cc(OC)c3s2)c2c(N)ncnn2c1CN1CCNC(=O)C1 ; "(I)"]

An alternate route from WO 2013/087578 leading to (I) is illustrated in scheme 2.

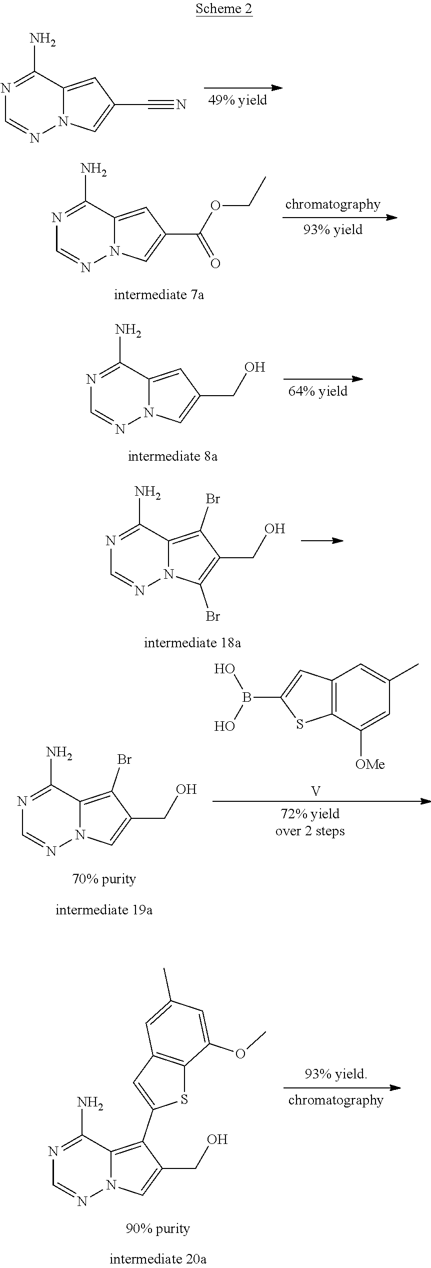

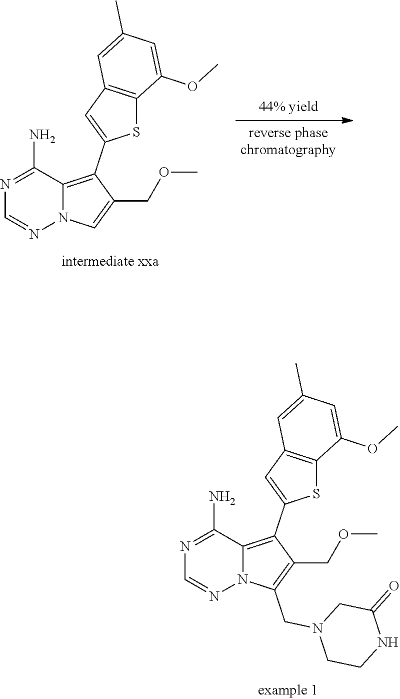 [SMILES: COCc1cn2ncnc(N)c2c1-c1cc2cc(C)cc(OC)c2s1 ; COCc1c(-c2cc3cc(C)cc(OC)c3s2)c2c(N)ncnn2c1CN1CCNC(=O)C1]

Preparation of 4-aminopyrrolo[2,1-f][1,2,4]triazine-6-carbonitrile is described in WO2007/064883 and is depicted in Scheme 3.

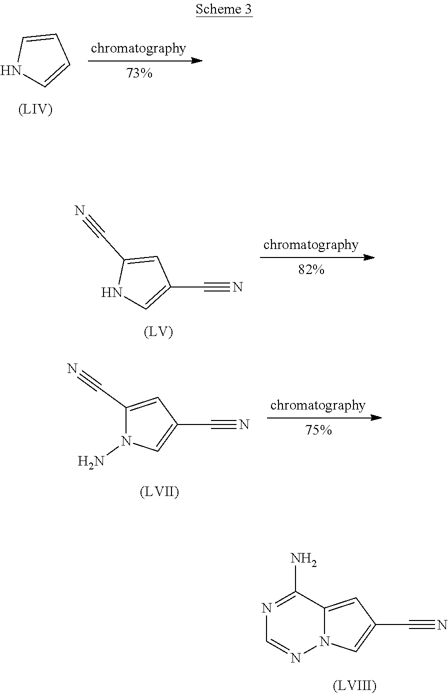 [SMILES: c1cc[nH]c1 ; N#Cc1c[nH]c(C#N)c1 ; N#Cc1cc(C#N)n(N)c1 ; N#Cc1cc2c(N)ncnn2c1]

A generic route for the preparation of compounds of the formula (I) is described in WO 2013/087578, but has not been applied to the synthesis of (I). It is depicted in scheme 4.

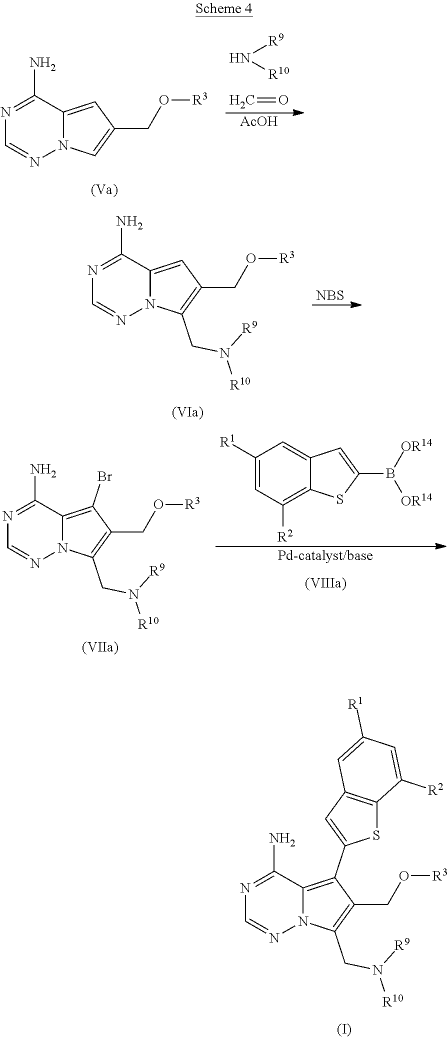

Preparation of intermediate (VII) has been described in WO 2013/087578 according to this generic route by the sequence shown in the following scheme 5. The total yield of this 4 step process from (IX) to compound (VII) was 6%, only and made use of 4 chromatographic purifications, which are unfavorable from an economic point of view. Further conversion of compound (VII) to (I) has not been described in prior art.

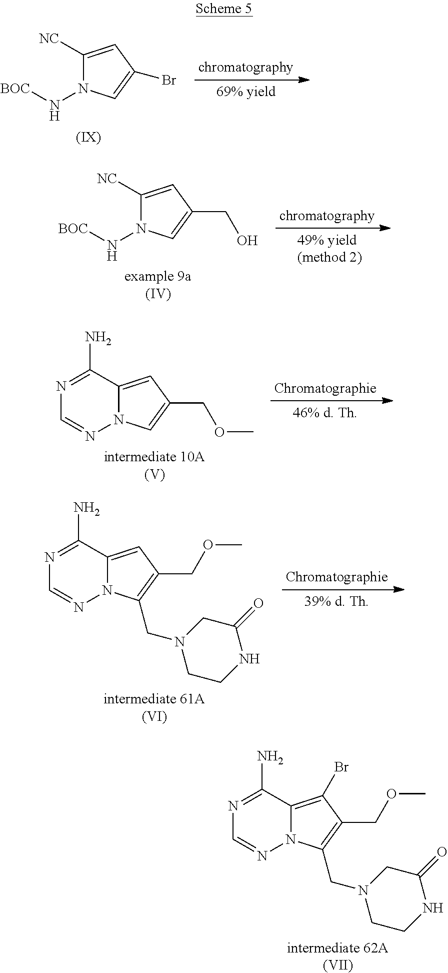

The preparation of (IX) has been described in WO 2007/064883 by the reaction sequence which is illustrated in Scheme 6.

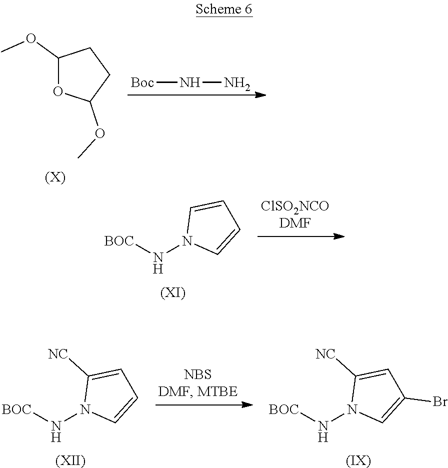

The di-hydrochloride of 4-{[4-Amino-6-(methoxymethyl)-5-(7-methoxy-5-methyl-1-benzothiophen-2-yl)pyrrolo[2,1-f][1,2,4]triazin-7-yl]methyl}piperazin-2-one (XIV) and its preparation process was disclosed first in WO 2013/087578A1 (Bayer)

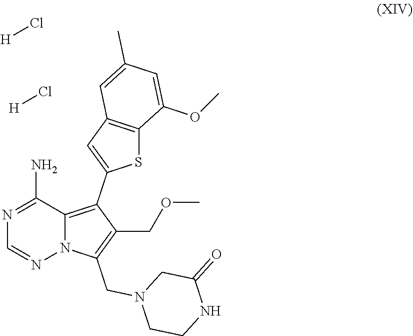 [SMILES: Cl ; COCc1c(-c2cc3cc(C)cc(OC)c3s2)c2c(N)ncnn2c1CN1CCNC(=O)C1 ; Cl]

The preparation of the di-hydrochloride was described in WO 2013/087578A1 (Bayer) example II. The only suitable method for obtaining this compound is by using HCl in dioxane. Other attempts to get the di-hydrochloride, e.g. by treatment with concentrated HCl in various solvents, results in non-isolable materials (highly hygroscopic; gums etc.). From a regulatory aspect, dioxane is not a favorable solvent to use in the final step of a synthesis, because the limit for residual solvent is very low. Also, ring-opened by-products from the reaction of HCl with dioxane can result in genotoxic impurities which have to be reduced to ppm level.

The di-hydrochloride is very hydroscopic and loses HCl on standing in the air (i.e., it is chemically unstable), which results in undefined mixtures of various hydrates and hydrochloride stoichiometry. It is very difficult to handle the di-hydrochloride on scale, especially on production scale.

The disadvantageous properties of the di-hydrochloride result in problems for large-scale preparations of the solid form of 4-{[4-Amino-6-(methoxymethyl)-5-(7-methoxy-5-methyl-1-benzothiophen-2-yl)pyrrolo[2,1-f][1,2,4]triazin-7-yl]methyl}piperazin-2-one. Thus, a need exists for stable salts and crystalline forms of rogaratinib.

While the processes disclosed by the prior art are per se effective for preparing the compound of the formula (I) and its synthesis intermediates, factors such as purity, product yields, process efficiency, safety and economy are very significant for an industrial scale process of a pharmaceutical product. A need also exists for an efficient process with high yield for preparation of compound of the formula (I) and its salts and various crystalline forms.

It is an object of the present invention to provide an efficient process with high yield for preparation of the compound of the formula (I)

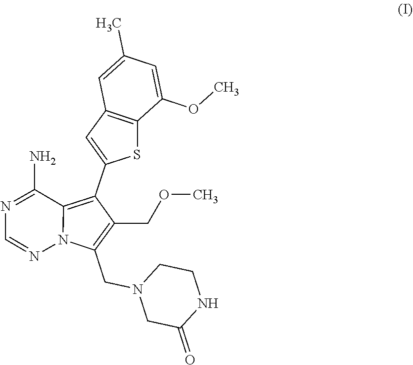 [SMILES: COCc1c(-c2cc3cc(C)cc(OC)c3s2)c2c(N)ncnn2c1CN1CCNC(=O)C1]

or a pharmaceutically acceptable salt, hydrate, or solvate thereof (rogaratinib).

It is an object of the present invention to provide a process for preparing the compound of the formula (I), in industrial scale (kilogram to metric tons range), which satisfies the criteria which apply in production and provides improvements in purity, environmental compatibility, industrial employability, safety aspects and volume yield. Purity and safety aspects are to be considered particularly relevant for the preparation of pharmaceuticals.

It is an object of the present invention to provide (I) in a solid state form which shows superior qualities compared to the known dihydrochloride.

The present invention solves those problems as described below.

### SUMMARY OF THE INVENTION

The present invention relates to compound (III)

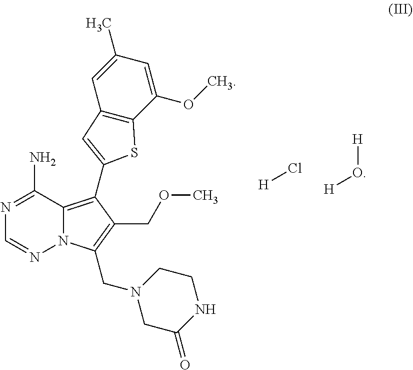

which is the monohydrate of the monochloride of compound (I)

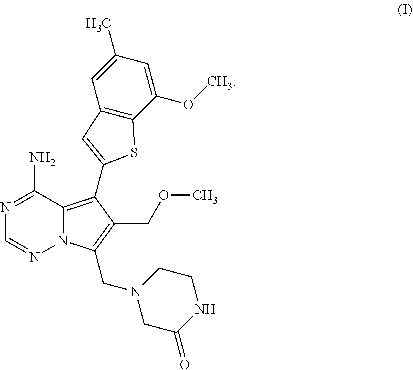 [SMILES: COCc1c(-c2cc3cc(C)cc(OC)c3s2)c2c(N)ncnn2c1CN1CCNC(=O)C1]

The present invention also relates to a pharmaceutical composition comprising monohydrate of the monochloride of compound I which is compound (III) and optionally further pharmaceutically acceptable excipients.

The present invention also relates to a process for preparing the compound of the formula (III), which is the monochloride monohydrate of compound (I), the process comprising suspending or dissolving (I) in the presence of a solvent and treating the resulting solution with an acid or acid precursor.

### DETAILED DESCRIPTION OF THE INVENTION

4-{[4-amino-6-(methoxymethyl)-5-(7-methoxy-5-methyl-1-benzothiophen-2-yl)pyrrolo[2,1-f][1,2,4]triazin-7-yl]methyl}piperazin-2-one corresponds to the formula (I), [4-{[4-amino-6-(methoxymethyl)-5-(7-methoxy-5-methyl-1-benzothiophen-2-yl)pyrrolo[2,1-f][1,2,4]triazin-7-yl]methyl}piperazin-2-one hydrochloride] corresponds to the compound of formula (II) and its monohydrate corresponds to the formula (III).

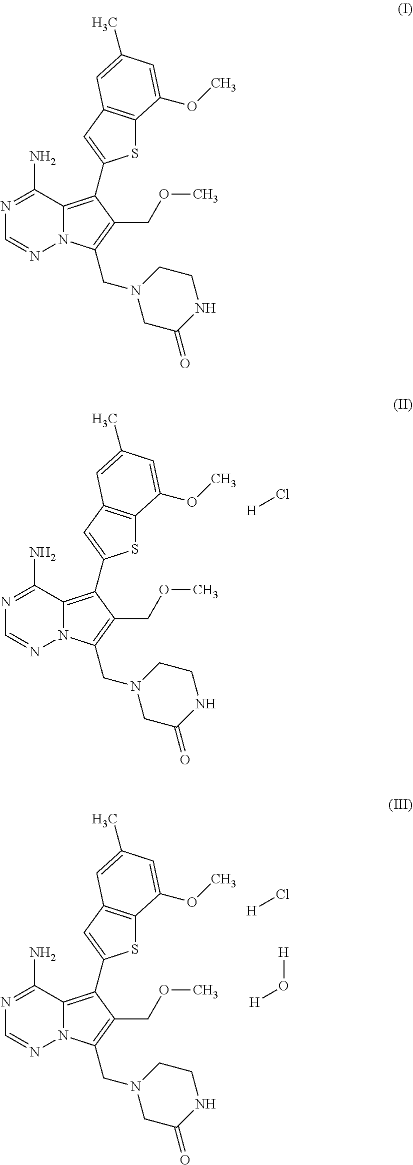 [SMILES: COCc1c(-c2cc3cc(C)cc(OC)c3s2)c2c(N)ncnn2c1CN1CCNC(=O)C1 ; COCc1c(-c2cc3cc(C)cc(OC)c3s2)c2c(N)ncnn2c1CN1CCNC(=O)C1 ; Cl ; COCc1c(-c2cc3cc(C)cc(OC)c3s2)c2c(N)ncnn2c1CN1CCNC(=O)C1 ; Cl ; O]

The inventive preparation of the compound of the formula (III), which is the monochloride monohydrate of compound (I) in its advantageous crystalline form, which previously has not been described, is shown in the following scheme:

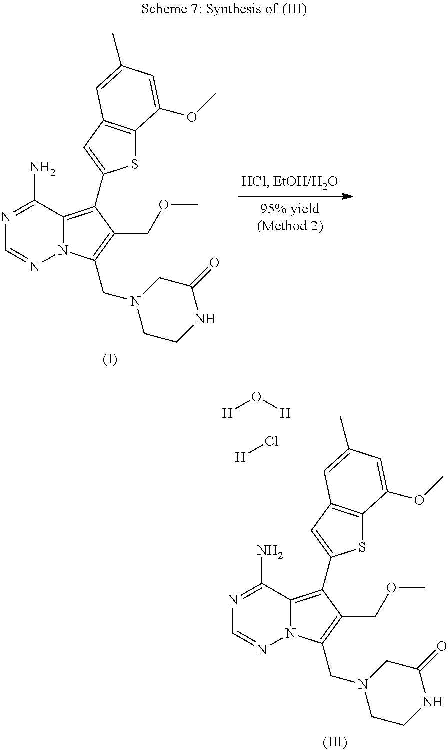

One aspect of the present invention is an efficient process with high yield for preparation of rogaratinib, which is obtained in very high purity without using chromatographic techniques. Furthermore, (I) is converted to its hydrochloride salt (II), more specifically as its crystalline monohydrate form with the chemical composition as in formula (III), having advantageous properties for using it as pharmaceutical ingredient.

### 4-{[4-Amino-6-(methoxymethyl)-5-(7-methoxy-5-methyl-1-benzothiophen-2-yl)pyrrolo[2,1-f][1,2,4]triazin-7-yl]methyl}piperazin-2-one-mono-hydrochloride Corresponds to the Compound of Formula (II)

The present invention provides the compound of formula (II) in a solid state which is
is physically and chemically stable
can be formulated as tablet without an undue burden
can be prepared in a reproducible manner, also on large scale
is easy to isolate, either by centrifuge or by filtration in high chemical purity
is easy to dry on scale
shows better solubility than the free base
is less hygroscopic than the Di-hydrochloride (prior art)
has good handling properties on scale, e.g., less electrostatic than the dihydrochloride
is easy to micronize and in high yields
is storable over a long period of time (important if you have only defined production slots over the year)

It has now been found that the monohydrate of the monohydrochloride of 4-{[4-Amino-6-(methoxymethyl)-5-(7-methoxy-5-methyl-1-benzothiophen-2-yl)pyrrolo[2,1-f][1,2,4]triazin-7-yl]methyl}piperazin-2-one [A] provides the benefits described above.

Along with the preferred new monohydrate form, several other new hydrates were found. Compound of formula (II) can exist in four different hydrate forms and an amorphous form. A ¾-hydrate (2.6% water), a monohydrate (3.5% water), a dihydrate (6.7% water) and a trihydrate (9.7% water) were found. The ¾-hydrate, the trihydrate as well as the amorphous form changed into the monohydrate during storage at high humidity. The dihydrate transformed into the monohydrate during storage in a closed vessel within two weeks. The following hydrate forms of compound II of the compound of formula (I) have been identified which are:
1. Monohydrate (one equ. water): A (Compound (III)
2. Dihydrate (two equ. water): B
3. Trihydrate (three equ. water): C
4. ¾ Hydrate (0.75 equ. Water): D
5. Amorphous Form: E

All together—the hydrate forms and the amorphous form—are different solid forms of the compound of formula (II).

Monohydrate of the compound of the formula (II) is preferred form and is referred to herein as compound (III). Surprisingly, compound (III) shows beneficial properties over the other solid forms of the compound of formula (II) with regard to:
physical stability: Storage at 25 and 50° C. for 12 month show no changes in stability;
chemical stability: The monohydrate is chemically stable during storage for several years
can be formulated as a tablet without undue burden by the process steps of dry blending, wet granulation, drying and dry milling, final blending, tablet compression and coating;
there is no interaction observed with the tablet ingredients. The monohydrate form is stable in the tablet matrix and does not change during storage (see Table 5)—compatibility over other ingredients is given;
can be prepared in a reproducible manner, also on large scale. This was demonstrated in several pilot plant campaigns, where >100 kg of drug substance were prepared;
is easy to isolate, either by centrifuge or by filtration. This was shown in several pilot plant campaigns. Isolation of monohydrate form is done without technical problems;
is isolated in high chemical purity and high chemical yield. This was shown in several pilot plant campaigns. The quality of the material is excellent and confirms the specification;
is easy to dry on scale. This was demonstrated in several pilot plant campaigns. The material can easily be dried under vacuum without significant losses of HCl and water. HCl and water values comply with the specification;
is more soluble than the free base. The monohydrate form has significantly better solubility in water. This results in an enhanced bioavailability;
is less hygroscopic than the di-hydrochloride. Moisture sorption tests (experimental part 4.4.) were performed with the monohydrate form. The compound was stored for 12 month at 15% r.h., 85% r.h and 97% r.h. (r.h.=relative humidity), no uptake of water was seen which clearly demonstrates that the monohydrate form is not hygroscopic. It is interesting to mention, that all other forms change into the monohydrate form under storage conditions (see Table 4.)
has good handling properties on scale and is less electrostatic. The monohydrate form is easily to handle in bulk. Weighing and pouring of the compound can easily be performed, no electrostatic properties were observed;
is easy to micronize and in high yields. Micronization of large batches were performed with yields generally >95% (th.). No problems during micronization were observed. The target particle size can easily be obtained in a reproducible manner;
is storable over a long period of time (important if you have only defined production slots over the year). Stability data demonstrate that the monohydrate compound form is very stable during storage;
the habitus of the crystals is acceptable in terms of filtration and isolation. Filtration times are very short which is a great advantage for handling in the pilot plant.

The monohydrate form (III) is therefore more suitable and preferred over the other solid forms of the compound of formula I for production on a large scale.

In particular, the compound of the formula (III) reduces any undesired conversion into another form of the compound of formula (II) and an associated change in the properties as described above is minimized. This should increase the safety and quality of preparations and formulations comprising of the compound of the formula (II) and the risk to the patient is reduced.

A pharmaceutical composition according to the present invention comprises compound (III) and optionally further pharmaceutically acceptable excipients.

Preferably, the pharmaceutical composition comprises compound (III), and no significant fractions of another form of the compound of the formula (II), and optionally further pharmaceutically acceptable excipients. More preferably, the pharmaceutical composition comprises more than 85 percent by weight, more preferably more than 90 percent by weight, most preferably more than 95 percent by weight, of compound (III) related to the total amount of all forms of the compound of the formula (II) present in the composition.

The different forms of the compound of formula (II) can be distinguished by X-ray powder diffraction, differential scanning calorimetry (DSC), IR-, Raman-, NIR-, FIR- and ¹³C-solid-state-NMR-spectroscopy.

The compound (III) of the compound of formula (I) can be characterized unambiguously by an X-Ray powder diffractogram (at 25° C. and with Cu—K alpha 1 as radiation source) which displays at least the following reflections: 9.3, 10.6, 13.3, preferably at least the following reflections: 9.3, 10.6, 13.3, 20.7, 23.3, more preferably at least the following reflections: 9.3, 10.6, 11.4, 13.3, 20.7, 23.3, 26.0, most preferably at least the following reflections: 6.8, 9.3, 10.6, 11.4, 13.3, 20.7, 23.3, 24.6, 26.0, 27.6, each quoted as 2θ value±0.2°.

The compound (III) can also be characterized unambiguously by the X-Ray powder diffractogram (at 25° C. and with Cu—K alpha 1 as radiation source) as shown in FIG. 1.

The dihydrate form [B] of the compound of formula (II) can be characterized unambiguously by a X-Ray powder diffractogram (at 25° C. and with Cu—K alpha 1 as radiation source) which displays at least the following reflections: 6.7, 13.9, 14.5, preferably at least the following reflections: 6.7, 11.7, 13.5, 13.9, 14.5, more preferably at least the following reflections: 6.2, 6.7, 11.7, 12.6, 13.5, 13.9, 17.9, most preferably at least the following reflections: 6.2, 6.7, 11.7, 12.6, 13.5, 13.9, 14.5, 16.4, 17.9, 25.9, each quoted as 2θ value ±0.2°. The compound of formula (I) in the dihydrate form [B] can also be characterized unambiguously by the X-Ray powder diffractogram (at 25° C. and with Cu—K alpha 1 as radiation source) as shown in FIG. 2.

The trihydrate form [C] of the compound of formula (II) can be characterized unambiguously by a X-Ray powder diffractogram (at 25° C. and with Cu—K alpha 1 as radiation source) which displays at least the following reflections: 6.8, 12.9, 14.6, preferably at least the following reflections: 6.8, 7.6, 12.9, 14.6, 26, more preferably at least the following reflections: 6.8, 7.6, 11.2, 12.9, 14.6, 22, 26.5, most preferably at least the following reflections: 6.8, 7.6, 11.2, 12.9, 13.5, 14.6, 17.4, 22.5, 23.3, 26.5, each quoted as 2θ value±0.2°. The compound of formula (I) in the trihydrate form [C] can also be characterized unambiguously by the X-Ray powder diffractogram (at 25° C. and with Cu—K alpha 1 as radiation source) as shown in FIG. 3.

The ¾-hydrate form [D] of the compound of formula (II) can be characterized unambiguously by a X-Ray powder diffractogram (at 25° C. and with Cu—K alpha 1 as radiation source) which displays at least the following reflections: 7.3, 12.2, 14.0, preferably at least the following reflections: 7.3, 12.2, 13.1, 13.4, 14.0, more preferably at least the following reflections: 7.3, 12.2, 13.1, 13.4, 14.0, 20.3, 22.4, most preferably at least the following reflections: 7.3, 12.2, 13.1, 13.4, 13.6, 14.0, 20.3, 21.2, 22.4, 26.3, each quoted as 2θ value±0.2°. The compound of formula (I) in the ¾-hydrate form [D] can also be characterized unambiguously by the X-Ray powder diffractogram (at 25° C. and with Cu—K alpha 1 as radiation source) as shown in FIG. 4.

Process for the Preparation of the Hydrogen Chloride Monohydrate (III)

One aspect of the present invention is directed to a process for the preparation of a monochloride salt (II), more specific as its crystalline monohydrate form with the chemical composition as in formula (III).

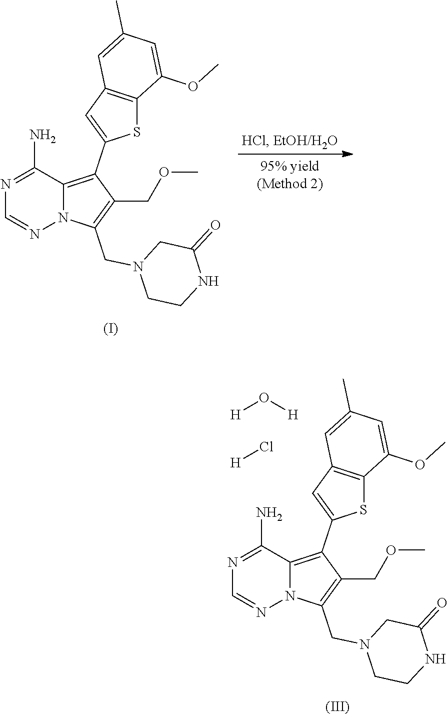

A general advantage of the invention compared to the state of the art processes is that it delivers the compounds (I) and (III) in satisfactory yields with very low impurity levels that match the requirements for APIs in late stage clinical development or market supply. The processes according to this invention can be carried out without making use of chromatographic purification steps. Furthermore, the state of the art processes have certain drawbacks which prevent application for industrial scale production, such as process safety concerns, product decomposition and enhanced impurity formation due to increased processing times upon scale-up, and limited through-put due to high dilutions. The inventive process described in the following can be used for large scale API production in standard industrial multi-purpose equipment for chemical synthesis without disproportional need for financial and personnel resources. This is achieved by optimized throughput and avoiding impurity formation by applying optimized and simplified processes and/or tailor-made purification processes on each stage of the synthesis. In summary a total yield of 36% was achieved with the final steps of the inventive process starting from (IV) to (III) as shown below:

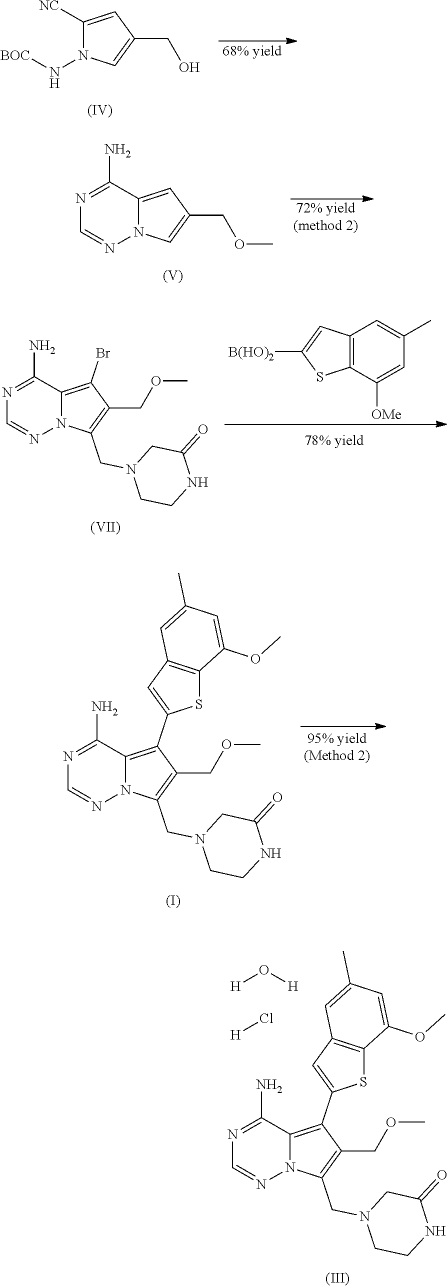

Direct comparison of the state of the art processes to (I) with the final synthetic steps of the inventive process is given in the following table:

| | | Isolated | | chromato- | |
|---|---|---|---|---|---|
| | | inter- | Overall | graphic | |
| Route | Starting material | mediates | yield | purifications | scale |
| Scheme 1 | (IV) | 4 | 0.8% | 4 prep. | labor- |
| | | | | chrom. | atory |
| | | | | 2 RP prep. | |
| | | | | chrom. | |
| Scheme 2 | 4- | 5 | 19% | 2 prep. | labor- |
| | aminopyrrolo[2, | | | chrom. | atory |
| | 1- | | | | |
| | f][1,2,4]triazine- | | | | |
| | 6-carbonitrile* | | | | |
| Inventive | (IV) | 3 | 36% | none | industri- |
| Process | | | | | al |

*long synthetic route to example 8a via 4-aminopyrrolo [2,1-f][1,2,4]triazine-6-carbonitrile, no common intermediate between Scheme 2 and Inventive Process

Method 1:

According to this aspect of the present invention, the conversion of (I) to (III) as shown above is carried out by suspending or dissolving (I) in the presence of a suitable solvent, preferably in water or alcohols, more preferably in a mixture of water miscible organic solvents with water, such as alcohols or ethers, most preferably ethanol or THF and treating it with hydrogen chloride or a hydrogen chloride precursor, most preferably hydrogen chloride.

Preference is given to initially charging the compound of the formula (I) to a solvent or solvent mixture and subsequently adding the acid, most preferably hydrogen chloride. Hydrogen chloride is added to this mixture, preferably as an aqueous solution, preferably at a temperature of between 20° C. and reflux conditions, more preferably at 40° C. to 60° C., more preferably at 45 to 55° C.

The reaction product is isolated by filtration and washed with water miscible organic solvents, such as alcohols or ethers, preferably ethanol. The product can be dried or submitted to next process steps without drying.

The product is then suspended in water or low concentrated aqueous hydrogen chloride solution, preferably 0.13% hydrogen chloride in water at elevated temperature to adjust the solid state form to the desired crystalline monohydrate form with the chemical composition as in formula (III). The mixture is cooled to 20±3° C. and isolated by filtration.

Compound (III) is dried preferably at a temperature of 50° C. and under reduced pressure, more preferably a pressure below 30 mbar without application of by-gas.

This process, referred to herein as “method 1 for preparation of compound (III),” has the advantage of transforming (I) into its monochloride, more specifically the monochloride monohydrate (III), which shows advantageous properties during application as an active pharmaceutic ingredient. Furthermore, this process has the advantage of reliably yielding the monochloride (II) as the monohydrate (III). Other forms, which may initially be formed during the salt formation step of the process, are transformed into the desired form during the treatment with dilute aqueous hydrogen chloride solution at elevated temperatures.

Method 2:

According to this aspect of the present invention the conversion of (I) to (III) is carried out by suspending or dissolving (I) in the presence of a suitable solvent and treating it with an acid or acid precursor. Preferably the solvent(s) are water or alcohols, more preferably a mixture of water miscible organic solvents with water, such as alcohols or ethers, most preferably ethanol or THF. The acid or acid precursor is preferably hydrogen chloride. Preference is given to initially charging the compound of the formula (I) to a solvent or solvent mixture and subsequently adding the acid.

Hydrogen chloride is added to this mixture, preferably as an aqueous solution, preferably at 20° C. to reflux conditions, more preferably at 40° C. to 60° C., most preferred at 45 to 55° C. A small aliquot, preferably 1 mass % related to the initial amount of (I), of monohydrate (III) (e.g. prepared by method 1), preferably having a fine particle size by prior milling or micronisation, is added to the suspension for the purpose of seeding in order to direct the product to the desired solid state form.

The reaction mixture is cooled down and the product is isolated on a filter dryer. The filter cake is washed with a water miscible organic solvent, preferably alcohols or ethers, most preferably ethanol or a mixture of ethanol and water. Then the filter cake is washed with water or low concentrated aqueous hydrogen chloride solution, preferably 0.13% hydrogen chloride in water at 20-35° C.

The product is dried under reduced pressure and elevated temperature, such as 30 mbar and 50° C. without application of by-gas.

This process, referred to herein as “method 2 for preparation of (III),” has the advantage of reliably forming (II) as the preferred monohydrate (III) immediately in the hydrogen chloride addition step, without manual handling of solid intermediates by performing reslurries or other unit operation to adjust to the desired solid state from. Surprisingly it was found, that the pseudopolymorphic form can be adjusted by adding seeding crystals into the suspension, after a hydrogen chloride salt of (I) has already been precipitated in other solid state forms. This seeding process has the advantage of improving filtration and drying properties on large scale compared to method 1 for preparation of (III).

This process has the advantage of strongly reducing the formation of impurities, such as (XV) and (XVI), which are formed during contact of (I) with acidic conditions:

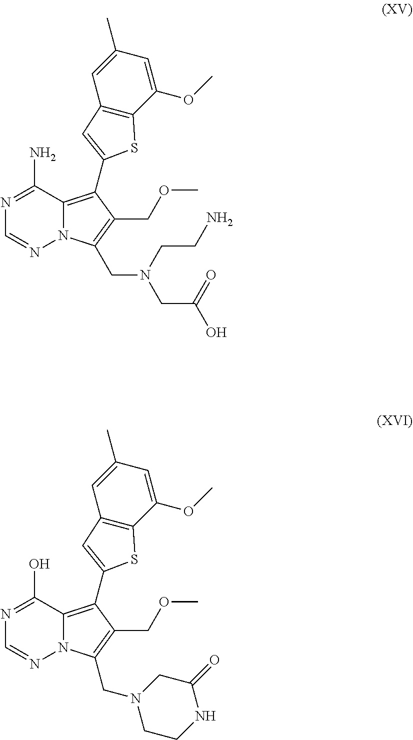 [SMILES: COCc1c(-c2cc3cc(C)cc(OC)c3s2)c2c(N)ncnn2c1CN(CCN)CC(=O)O ; COCc1c(-c2cc3cc(C)cc(OC)c3s2)c2c(O)ncnn2c1CN1CCNC(=O)C1]

This is achieved by minimizing processing and handling times—especially on large scale—especially by avoiding additional acidic treatments to adjust the product to the desired polymorphic form. This allows for achieving impurity levels that match the requirements for APIs in late stage clinical development or market supply.

According to the inventive process, potential side products, in particular the compounds of the formula (XV) and (XVI) and further, can be separated very effectively from (III) because these side products or their salts do not precipitate under the conditions according to the present process and remain in the filtrate.

Another embodiment of the present invention is the compound of formula (III), substantially free of palladium, with palladium present in an amount up to 100 ppm, preferably up to 60 ppm, most preferably 0-2 ppm, and in a very high purity containing one or more pyrrolo-triazine substances structurally related to (I) each from 0% to a maximum of 0.15%, preferably each from 0% to a maximum 0.06% HPLC area % based on the amount of the compound of the formula (I). Pyrrolo-triazine substances structurally related to (I) include but are not limited to the compounds of the formula (XV), (XVI), (XVII), (XVIII) and (VI).

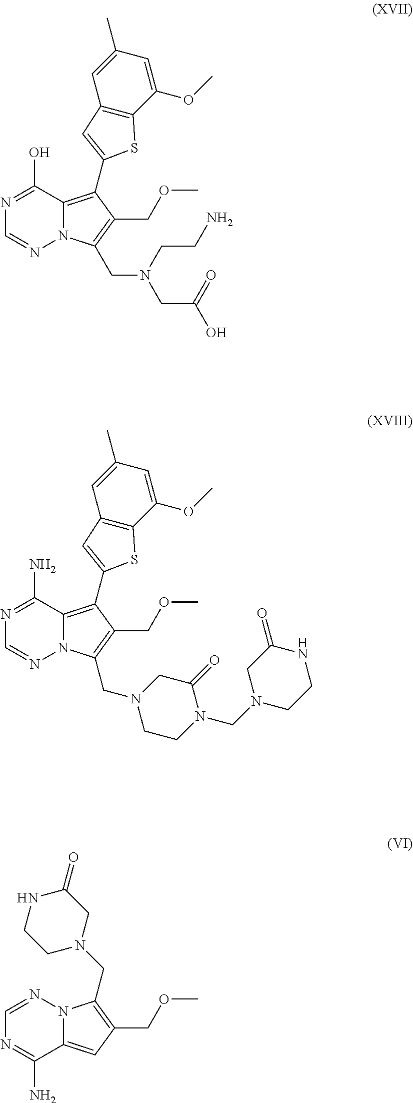 [SMILES: COCc1c(-c2cc3cc(C)cc(OC)c3s2)c2c(O)ncnn2c1CN(CCN)CC(=O)O ; COCc1c(-c2cc3cc(C)cc(OC)c3s2)c2c(N)ncnn2c1CN1CCN(CN2CCNC(=O)C2)C(=O)C1 ; COCc1cc2c(N)ncnn2c1CN1CCNC(=O)C1]

Preparation of the Compound of the Formula (I)

One aspect of the present invention relates to a process for preparing the compound of the formula (I) may be prepared by reacting the compound of the formula (VIIb), wherein R1 is halogen or other suitable leaving group), most preferably bromine, with the compound of the formula (VIIIb) wherein R2 is a suitable metalorganic substituent such as Li, MgR, Sn, and B, carboxylic acid, hydrogen, or boron derivatives, such as boron-esters, boron-amides, MIDA, preferably hydrogen or boron derivatives, most preferably boronic acid in the presence of a suitable catalyst. Substituent R2 may also comprise hydrogen in catalytic C—H activation reactions leading to the compound of the formula (I).

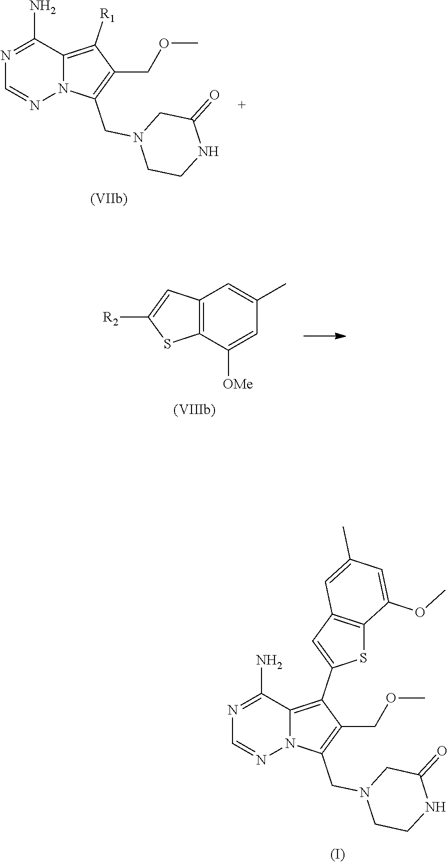

A mixture of (VIIb) and (VIIIb) is treated in the presence of a base such as hydroxides, (hydrogen-) carbonates, fluorides, or amines, in a suitable organic solvent or mixture with water, at elevated temperatures with a transition metal catalyst, preferably with a suitable palladium catalyst.

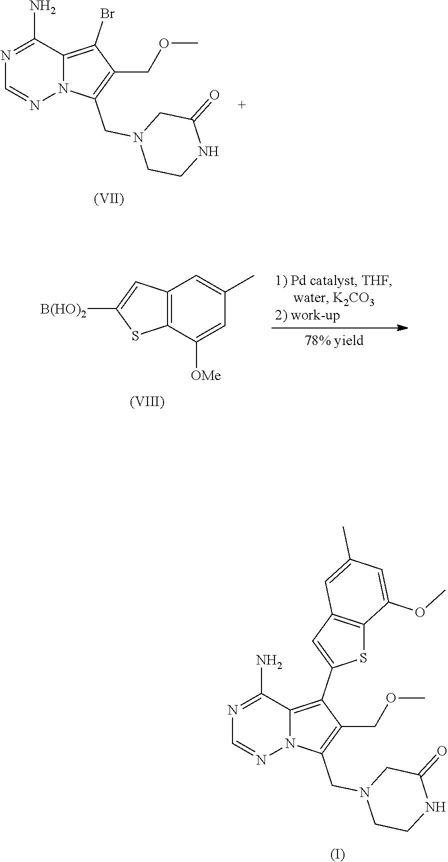

Preference is given to treating a mixture of the compounds of the formulas (VII) and (VIII) in THF and water with K₂CO₃ as a base and a catalyst at a temperature of 60° C. to reflux for 30 min to 300 min.

Suitable palladium catalyst are, but are not limited to: X-Phos precatalyst=Chloro(2-dicyclohexylphosphino-2′,4′,6′-triisopropyl-1,1′-biphenyl)[2-(2′-amino-1,1′-biphenyl)]palladium(II) and

Pd(dbpf)Cl2=[1,1′-Bis(di-tert-butylphosphino)ferrocene]dichloropalladium(II) and

PdCl2(Amphos)2=Bis(di-tert-butyl(4-dimethylaminophenyl)phosphine)dichloropalladium(II).

This process yields a mixture of the compound of the formula (I) with by-products and remaining reagents referred to as the crude reaction mixture. This crude reaction mixture can be processed by the following method:

Another aspect of the present invention is a process to obtain a solid and purified version of the compound of the formula (I). The process comprises treating the crude reaction mixture by addition of an aqueous solution of a palladium-scavenging reagent, such as acetyl cysteine at a temperature from 20° C. up to reflux temperature, most preferably at 60° C. for 1 h up to 24 h. The solvent used during the reaction, such as THF, can be removed by distillation, optionally under reduced pressure. A suitable solvent, preferably a non-water miscible solvent that readily extracts traces of (XIX), most preferably MTBE or EtOAc can be added before or after the distillation. After cooling, preferably to a temperature of from 0° C. to 30° C., preferably 20° C., the compound is isolated by filtration. This purified compound (I) can be submitted to further purification processes.

In order to provide a highly purified version of the compound of the formula (I), it is charged into a suitable organic solvent or solvent mixtures and heated to elevated temperatures, most preferably a mixture of THF and water or ethanol and water at temperatures such as 50° C. to reflux. The compound of the formula (I) is isolated by filtration at a temperature above −10° C. and below reflux temperature, preferentially between 0° C. and 20° C.

Preference is given to charging the compound of the formula (I) in a mixture of tetrahydrofuran with water in a ratio of 85 volumes tetrahydrofuran to 15 volumes of water and heating the mixture until a solution is obtained. THF is removed by distillation, preferentially under reduced pressure and ethanol is added in order to change the solvent composition to mainly comprise ethanol and water. The mixture is cooled to 15° C. within and the compound of the formula (I) is isolated by filtration. This purification procedure can be repeated to further reduce impurity levels.

The compound is dried under reduced pressure and at elevated temperature.

Potential side products, in particular traces of palladium, benzothiophenylic side products, such as (XIX):

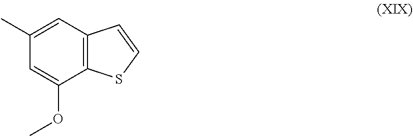

and one or more pyrrolo-triazine substances structurally related to (I) such as the starting compound (XII), (VI) and (XVIII) do not precipitate under the conditions according to the present process and remain in the filtrates.

Another embodiment of the present invention is the compound of formula (I) in a very high purity containing one or more pyrrolo-triazine substances structurally related to (I) each from 0% to a maximum of 0.15%, preferably each from 0% to a maximum 0.06% by HPLC area % based on the amount of the compound of the formula (I). Pyrrolo-triazine substances structurally related to (I) include but are not limited to the compounds of the formula (XII), (VI) and (XVIII).

Another embodiment of the present invention is the compound of formula (I) in a very high purity containing traces of palladium determined by appropriate trace methodology from 0 ppm to a maximum of 60 ppm, typically below 2 ppm.

Preparation of the Compound of the Formula (VII)

Another aspect of the present invention is a process for preparing the compound of the formula (VIIb), wherein R1 can be chlorine, bromine or iodine, most preferred bromine, by reacting compounds of the formula (V) and (XIII) with paraformaldehyde in the presence of an acid to an intermediate product of the formula (VI). The product of formula (VI) is not isolated, but treated with a halogenation agent, such as a bromination, iodination, or chlorination agent. Preferably a bromination agent is used, most preferably N-Bromo Succinimide (NBS) in the same reaction vessel as a one-pot reaction. While intermediate (VI) is difficult to isolate and purify, especially using standard industrial operations on larger scale, the brominated derivative with the chemical structure (VII) crystallizes readily from the reaction mixture in good purity. Purity can be further enhanced by suspending (VII) in suitable solvent or solvent mixtures at elevated temperatures.

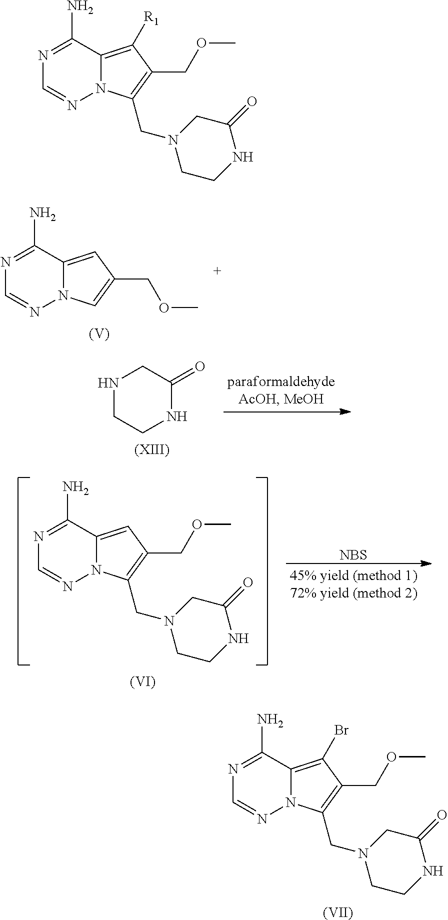

In a preferred embodiment of the process for preparation of the compound of the formula (VII) the compounds of the formula (V) and (XIII) are charged into a suitable solvent, preferably methanol, ethanol, iso-propanol, n-propanol, n-butanol and their mixtures with water, most preferred in MeOH.

A source of formaldehyde, preferably paraformaldehyde, formalin solutions, or other formaldehyde sources, most preferably paraformaldehyde, an acidic agent, preferably carboxylic acids, such as acetic acid, benzoic acid, propionic acid, trifluoro acetic acid, sulfonic acids, such as p-toluene sulfonic acid, benzene sulfonic acids, mineral acids, such as hydrogen chloride, sulfuric acid, phosphorous acid, most preferably acetic acid and heated to elevated temperature, preferably 40-100° C., most preferably to 60° C. to reflux for 1 h to 48 h, preferably for 20-24 h.

1 eq to 4 eq piperazin-2-one (XIII), 1 eq to 3 eq paraformaldehyde and 1 eq to 10 eq of acetic acid are deployed in the reaction. Preferably 1 eq to 2 eq piperazin-2-one (XIII), 1 eq to 1.5 eq paraformaldehyde and 3 eq to 7 eq of acetic acid are deployed in the reaction. Most preferably 1.5 eq piperazin-2-one (XIII), 1.1 eq paraformaldehyde and 6 eq of acetic acid are deployed in the reaction.

After conversion to (VI), an additional suitable solvent, such as protic and aprotic organic solvents and water can optionally be added with or without combination with a inorganic or organic base, such as triethyl amine, pyridine, Hünig's base, 2,6-lutidine, N-methyl imidazole, or inorganic bases, such as sodium hydroxide, potassium hydroxide, calcium hydroxide, sodium carbonate, or potassium carbonate.

Most preferably, an aqueous solution of sodium hydroxide is added until a slightly acidic or neutral pH is reached. Surprisingly an optimum between best conversion, limited impurity formation, good stirring properties, and enhanced isolation properties by reduced fine particle formation can be achieved by applying a pH of 5.5 to 6.5 during bromination.

The bromination agent, preferably NBS or 1,3-dibromo-5,5-dimethylhydantoin (DBDMH), most preferably NBS, is added as a solid or as a solution in a suitable solvent, preferably acetonitrile. It is advantageous to add solid NBS in portions or by slow addition of a solution of NBS in acetonitrile, to reduce impurity formation.

The bromination is carried out at −20° C. to 20° C., preferably at −10° C. to 10° C., most preferred at −8° C. to −2° C. It is advantageous to heat the reaction mixture to reflux and cool down again after the reaction is finished, to improve isolation.

In order to provide a highly purified version of the compound of the formula (VII), the reaction product is charged into a suitable organic solvent or solvent mixtures, preferably alcohols, ethers, nitriles, water and mixtures thereof, most preferably methanol, THF and mixtures of methanol and THF with water and heated to elevated temperatures such as 50° C. to reflux. The compound of the formula (VII) is isolated by filtration at a temperature above −10° C. and below reflux temperature, preferentially between 0° C. and 20° C. The filter is finally washed with water or a mixture of a solvent, mixed with water, preferably MeOH or THF. Most preferable a mixture of MeOH and water.

In order to prepare the compound of the formula (VII), the filtered and washed product is dried, preferably at ambient temperature and optionally under reduced pressure. The compound of the formula (VII) is obtained as a hydrate containing approximately 5% of water.

Potential side products, in particular side products such as the compound of the formulas (XX), (XXI) and (VI) do not precipitate under the conditions according to the present process and remain in the filtrates.

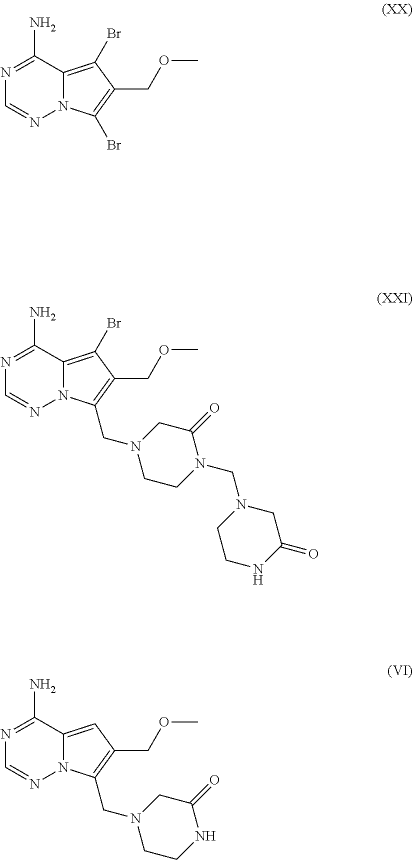 [SMILES: COCc1c(Br)c2c(N)ncnn2c1Br ; COCc1c(Br)c2c(N)ncnn2c1CN1CCN(CN2CCNC(=O)C2)C(=O)C1 ; COCc1cc2c(N)ncnn2c1CN1CCNC(=O)C1]

Another embodiment of the present invention is the compound of formula (XII) in a very high purity. Side products include but are not limited to (XX), (XXI) and (VI) in amounts such as: (XX) from 0% to 0.50%, preferably 0% to 0.30%, (XXI) from 0% to 0.70%, preferably 0% to 0.30%, and (VI) from 0% to 0.30%, preferably 0% to 0.20% by HPLC area % based on the amount of the compound of the formula (VII).

Preparation of the Compound of the Formula (V)

Another aspect of the present invention is a process for preparing the compound of the formula (V)

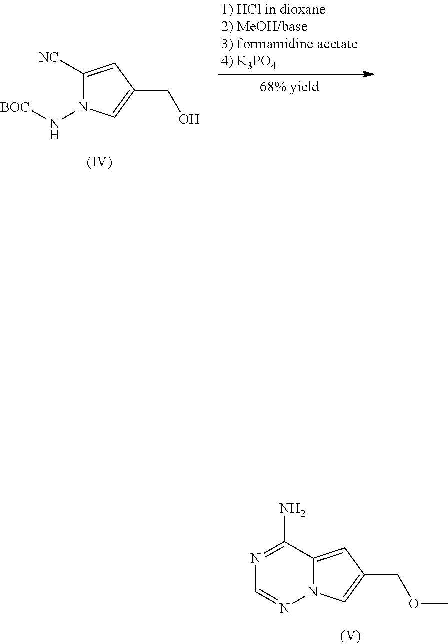

from the compound of the formula (IV) via the reaction intermediates of the formulas (XXII) and (XXIII),

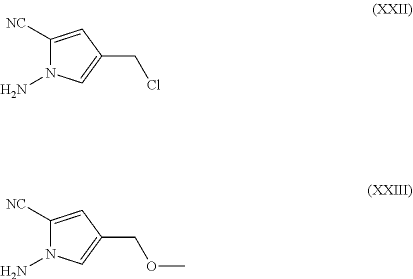

by a reaction sequence of acidic cleavage of the BOC substituent, chlorination and etherification of the alcohol moiety, and cyclisation with a reagent containing formamidine with or without isolation of intermediates.

In a preferred embodiment of the process for preparing of the compound of the formula (XII), the compound of the formula (V) is charged into a solution of a suitable acid, in a suitable solvent until intermediate (XXII) is formed. The reaction mixture is then reacted with methanol or alkali methylate with or without the presence of a suitable base to form the reaction intermediate (XXIII). Then formamidine or a formamidine precursor is added and the mixture is heated to elevated temperature, preferably between 40° C. and reflux, most preferably to 60-66° C. Conversion to (V) can be completed by a adding a base preferentially as an aqueous solution.

In the process for preparing of the compound of the formula (V), the compound of the formula (VII) is charged into to a solution of 13-14% HCl in dioxane at 19° C. to 25° C. After completion of the conversion to intermediate (XXII), typically about 6 h, the reaction mixture is charged into a mixture of methanol and a suitable base, such as K₃PO₄, alkali methylates, inorganic carbonates, inorganic hydrogencarbonates, hydroxides, organic amine bases, preferably 1 eq to 2 eq K₃PO₄ or 2 eq to 3 eq of sodium methylate, most preferably 2.5 eq of sodium methylate at a temperature of 20° C. to 30° C. and stirred until complete conversion to the intermediate (XXIII), typically for 1 h. Then formamidine or a formamidine precursor, most preferred 6 eq of formamidine acetate are added to the reaction mixture and the mixture is heated to 55° C. to reflux (approx. 67° C.) until complete conversion of the intermediate (XXIII), typically for 16 h to 20 h. An aqueous solution of a suitable base such as K3PO4, alkali methylates, inorganic carbonates, inorganic hydrogencarbonates, hydroxides, organic amine bases, most preferred 4 eq K₃PO₄ is added and the mixture is heated to 55° C. to reflux (67° C.) until complete conversion to the compound of the formula (V), typically for 2 h. The organic solvents are removed by distillation, preferentially under reduced pressure, and iso-propyl acetate is added. The aqueous and the organic phases are separated, preferably at a temperature of 45° C., and the aqueous phase is extracted with iso-propyl acetate, preferably at a temperature of 45° C. The combined organic phases are concentrated by distillation, preferentially at moderate temperatures under reduced pressure. The resulting suspension is heated to 80° C. until most of the product is dissolved again and slowly cooled to 0° C. to 20° C. The product is isolated by filtration. In order to prepare the compound of the formula (XII) it is dried preferably at a temperature of 40° C. to 60° C. and optionally under reduced pressure.

The process has the general advantage of avoiding impurity formation. Specifically when the acidic solution of intermediate (XXII) is reacted with methanol without the presence of a base, then the side component of the formula (XXIV) is found in the product (V) of the process. The level of the side component of the formula (XXIV) in the product (V) is depending on the time for this process step. In a typical reaction at pilot plant scale of converting 120 kg (VII) with a time of 1 h for this process step, around 11% of (XXIV) are formed, leading to up to 7% of this impurity in the final product. By charging the acidic solution of intermediate (XXII) into a solution of methanol with a suitable base, the formation of the side components of the formula (XXIV) and (XXV) can be strongly reduced.

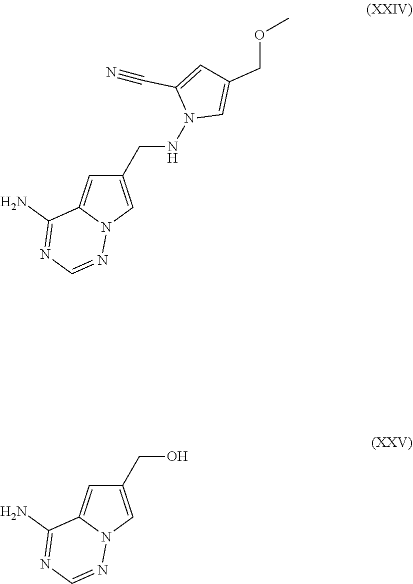 [SMILES: COCc1cc(C#N)n(NCc2cc3c(N)ncnn3c2)c1 ; Nc1ncnn2cc(CO)cc12]

Furthermore this process leads to strongly reduced processing times and strongly reduced tar formation especially on industrial scale, thus avoiding increased effort for work-up, purification and isolation of (V). This is achieved by applying a limited amount of base, during the reaction of intermediate (XXII) with methanol, so that strongly basic conditions are avoided during the conversion with formamidine. Therefore, decomposition of reagents can be reduced. By adding an excess of base as an aqueous solution after reaction with formamidine, the complete conversion to (V) is triggered and residual formamidine side products thereof, are immediately removed to the aqueous face, before excessive tar formation occurs.

According to the present process potential side products, in particular side products, such as the compound of the formulas (XXIII), (XXIV) and (XXV) do not precipitate under the conditions according to the present process and remain in the filtrates.

Another embodiment of the present invention is the compound of formula (V) in a very high purity. Side products include but are not limited to (XXIII), (XXIV) and (XXV) in amounts such as: (XXIII) from 0% to 0.15% and (XXIV) from 0% to 0.15% and (XXV) from 0% to 0.15% by HPLC area % based on the amount of the compound of the formula (I).

Another embodiment of the present invention is a recrystallization process for the purification of the compound of the formula (V). If (V) is not produced by the inventive process described here, (V) can be obtained in reduced quality, e.g. containing high amounts of side components and salts, and with low assay for use. In order to improve the quality of such samples, (V) an be recrystallized by dissolving it in mixtures of alcohols with aprotic solvents, preferably in a mixture of ethanol with isopropyl acetate at elevated temperatures up to reflux and slowly cooling down again. The purified compound (V) can be isolated in good yield and high purity.

Preparation of the Compound of the Formula (IV)

Another aspect of the present invention is a process for preparing the compound of the formula (IV)

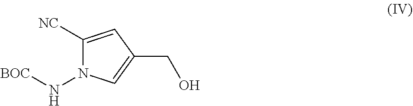 [SMILES: CC(C)(C)OC(=O)Nn1cc(CO)cc1C#N]

The reaction sequence depicted in scheme 8 outlines a preparation of the compound (IV) via the intermediates (XI), (XII) and (IX). It generally follows the synthetic sequence in analogy to WO 2007/064883 until the intermediate (VII), and the conversion of compound (VII) to (VII) is done in analogy to the process described in WO2013/087578. Compared to the state of the art processes, the inventive process delivers (IV) by improved methods and processes for the efficient and safe production on industrial scale and without chromatographic purification steps.

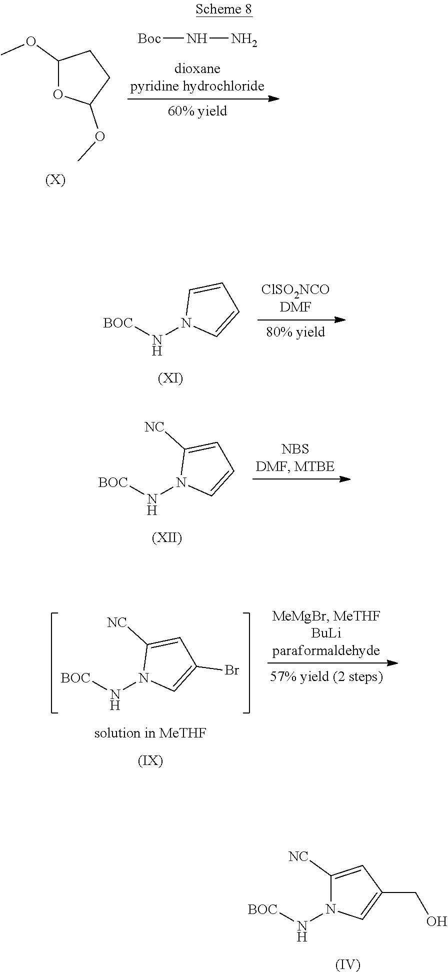

In the process for the preparation of the compound of the formula (XI), 2,5-dimethoxytetrahydrofuran (X) is reacted with tert-butyl hydrazinecarboxylate in the presence of pyridine hydrochloride in a solvent mixture of dioxane and pyridine at a temperature of 102±3° C. Under these conditions methanol formed during the reaction is removed by distillation. After complete conversion water and a non-water miscible organic solvent preferably di-nbutyl ether are added and the product can be isolated from this mixture. The inventive process to compound (XI) has been applied to large scale production and has the advantage of reduced formation of side components, especially on large scale, by using pyridine and pyridine hydrochloride salts as reagents.

The compound of the formula (XII) is prepared by reacting compound (XI) with chlorosulfonyl isocyanate in DMF. The crude product can be isolated by addition of the reaction mixture into an aqueous solution of an inorganic salt, such as hydroxides and carbonates, most preferably ammonium hydrogen carbonate, followed by filtration. The crude product is purified by dissolving it in a suitable organic compound, preferably methanol, and the product is precipitated by mixing the solution with water. The inventive process to compound (XII) has been applied to large scale production and has the advantage of delivering (XII) in good purity without chromatographic purification.

The compound of the formula (IX) is prepared by reacting compound (XII) with N-bromosuccinimide in a mixture of DMF and methyl tert-butyl ether. After hydrolysis of the reaction mixture, the product is extracted with methyl tetrahydrofuran and the solution of compound (IX) in methyl tetrahydrofuran is submitted to the next stage without isolation or purification. The inventive process to compound (IX) has been applied to large scale production and has the advantage simplifying the process by avoiding isolation of (IX) as a solid and telescoping it into the preparation of (IV).

The compound of the formula (IV) is prepared by reacting compound (IX) with metal organic reagents, preferably with methyl magnesium bromide and butyl lithium and addition to paraformaldehyde. Variations of yield and quality have been observed, in dependence to different batches of paraformaldehyde. This was overcome by treating paraformaldehyde with methyl tetrahydrofuran prior to use. The purified compound of the formula (IV) is obtained after hydrolysis and crystallization. The inventive process to compound (IV) has been applied to large scale production and has the advantage of delivering (IV) in good purity without chromatographic purification.

Preparation of the Compound of Formula (VIII)

This preparation is described in European Patent Application No. 15180755.9, the entire contents of which are hereby incorporated by reference. A preferred method is described in detail below:

Step 1:

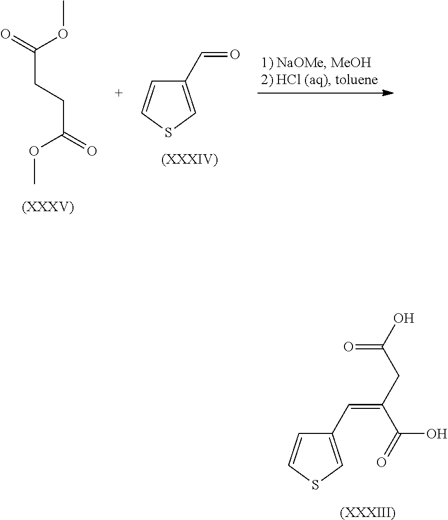

The reaction of (XXXV) and (XXXIV) to (XXXIII) as shown above is carried out by condensation of (XXXV) with (XXXIV). This is done by adding a solution of an alkali alcoholate, such as sodium methanolate, in an alcohol, preferably methanol to a solution of dimethyl succinate at 25-40° C. Other succinate esters can be used in place of (XXXV), as the esters are cleaved during following steps.

The mixture is heated to reflux and a solution of thiophene-3-aldehyde is added. After complete conversion the mixture is hydrolyzed by addition of water and the product is extracted with toluene. (or other non-water miscible solvents) After removal of the solvent the crude (XXXIII) is purified by crystallization and/or reslurry from toluene (or other suitable solvents).
This process has the advantage of high conversion related to the aldehyde by slow addition of the thiophene-3-aldehyde to the reaction mixture.
This process has the advantage of applying reduced excess of dimethyl succinate for full conversion.
This process has the advantage of giving a very pure and solid intermediate (XXXIII) after purification by crystallization or/reslurry, contributing to avoidance of purification on later stages by e.g. preparative chromatography.

Step 2:

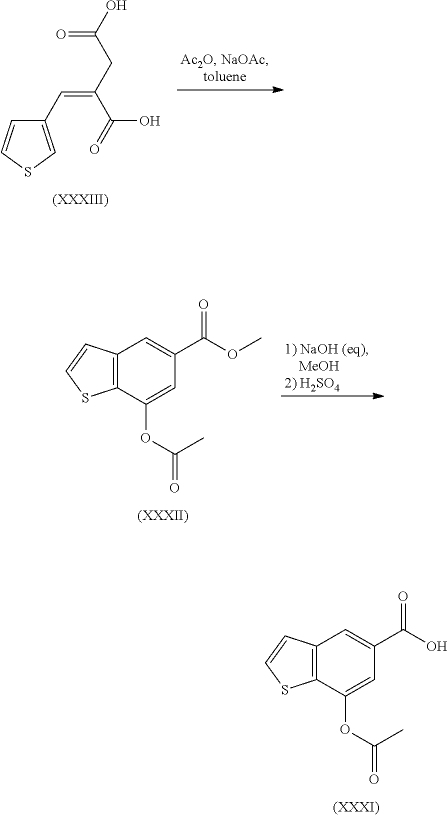

The reaction of (XXXIII) to the carboxylic acid intermediate (XXXI) via (XXXII) as shown in Step 2 is carried out by ring-closure to the benzothiophen derivative (XXXII) under dehydrating conditions and hydrolysis of the ester moieties yielding the 7-hydroxy-1-benzothiophene-5-carboxylic acid (XXXI). This is done by heating (XXXIII) with acetic acid anhydride and sodium acetate in toluene at 70-75° C. for 7 h (other dehydrating agents: e.g. acid anhydrides (trifluoracetic acid anhydride), methyl chloro formate; other bases than sodium acetate (potassium acetate; T & t can be varied for all process steps). The mixture is hydrolyzed by addition of water at 25-30° C. The organic phase is separated, washed with water, again, and the solvent is partially removed by distillation under reduced pressure. The remaining solution of (XXXII) in toluene is diluted with MeOH and water and an aqueous sodium hydroxide solution (other bases, mainly inorganic) is slowly added at temperatures below 45° C. and finally heated to 50-55° C. for 5 h. The aqueous phase is separated and further diluted with water and the product is precipitated by addition of a strong protic acid such as HCl, HNO₃, sulfonic acids, CH₃COOH and H₂SO₄, preferably H₂SO₄ at 10-15° C. till a pH of 2-3 is reached. The suspension is heated to 40-45° C. and cooled to 25-30° C. within 2 h to improve filtration behavior of the product, and isolated by filtration.
This process has the advantage of increased process safety for industrial scale by not using a large excess of acetic acid anhydride as a solvent, but a limited excess by dilution in toluene. Safe work-up is achieved by controlled release of energy during hydrolyzation of acetic acid anhydride under diluted conditions.
This process has the advantage of giving reduced amounts of side products by using only moderate reaction temperatures during the ring closure step towards (XXXII).
This process has the advantage of acceptable filtration times on industrial scale during isolation of (XXXI) by improving solid state properties during temperature treatment before isolation.
This process has the advantage of yielding a well crystalizing solid product of intermediate (XXXI) with very high purity in very good yield, avoiding additional purification steps on intermediate (XXXII) or later stages of the synthesis.

Step 3:

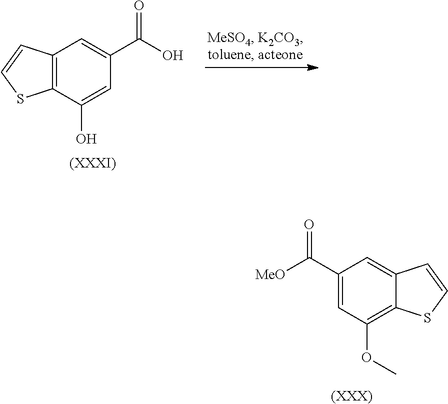

The reaction of (XXXI) to methyl 7-methoxy-1-benzothiophene-5-carboxylate (XXX) as shown in scheme is carried out by methylating the ester and phenol moiety. This is done by dissolving (XXXI) in a mixture of acetone and toluene (other solvents). After addition of a potassium carbonate (other bases inorganic, amines) the suspension is heated to 50-60° C. and dimethylsulfate (other methylating agents: methyl iodide) is slowly added. After full conversion the solvent is partially distilled of at 85° C. and water is added. Phases are separated and aqueous phase is additionally extracted with toluene. Combined organic phases are washed with water and the solvent is removed under reduced pressure at 60° C. The crude product is submitted to the next step.

Step 4:

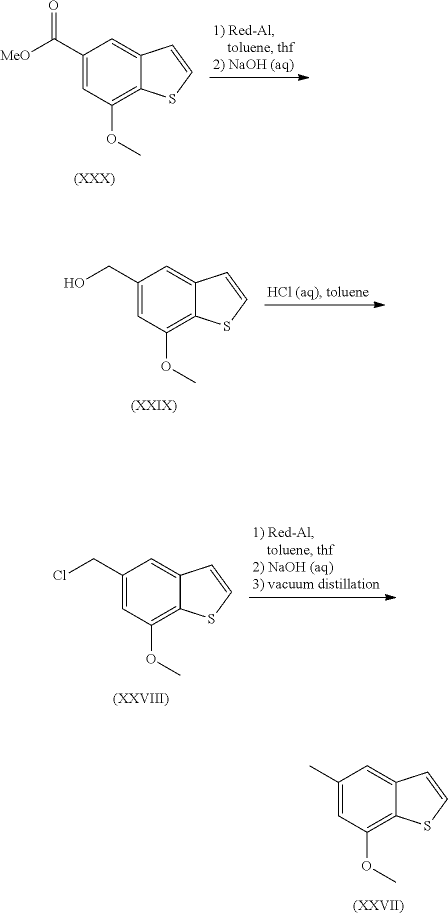

The reaction of (XXX) to 7-methoxy-5-methyl-1-benzothiophene (XXVII) is done by reduction of the ester moiety to the methyl group yielding (XXVII). This is preferentially achieved by stepwise reduction through reducing the ester moiety of (XXX) to the alcohol (XXIX), followed by chlorination of the alcohol moiety to (XXVIII), followed by reduction to (XXVII) as shown in Step 4. This is done by dissolving the crude product (XXX) in an inert solvent such as ethers, for example dioxane Me-THF, CPME, and MTBE, aromatic & aliphatic hydrocarbons, for example benzene, toluene, xylol cyclohexane; preferably THF is used and addition of sodium-bis(2-methoxy-ethoxy)-aluminium-dihydride (Red-AlR) solution in toluene at 25-30° C. Other suitable reducing agents include hydrogen (with a suitable catalyst), LAH, boranes and silanes.

The mixture is hydrolyzed by addition of aqueous sodium hydroxide solution (other aqueous bases) and the product is extracted with toluene (other no-water miscible solvents or precipitated/crystallized by anti-solvent addition) and isolated by removing the solvent under reduced pressure at 60° C.

Crude (XXIX) is dissolved in toluene and at 50-55° C. aqueous HCl is slowly added. Other chlorinated agents such as SOCl₂ may be utilized. After complete conversion the mixture is hydrolyzed with aqueous sodium bicarbonate solution. The organic phase is dried by treatment with brine, Na₂SO₄ and azeotropic drying by removing the solvent under reduced pressure at 60° C.

Also, other leaving groups can be used as an alternative chlorine in structure (XXVIII), such as Br, I, F, RSO₃, for example.

Crude product (XXVIII) is dissolved in an inert solvent such as ethers, for example Dioxane Me-THF, CPME, and MTBE, aromatic & aliphatic hydrocarbons, for example benzene, toluene, xylol cyclohexane; preferably THF is used and a reduced using a reducing agent such as sodium-bis(2-methoxy-ethoxy)-aluminum-dihydride (Red-Al®) solution in toluene is added at 25-30° C. Other suitable reducing agents include hydrogen (with a suitable catalyst), LAH, boranes and silanes.

The mixture is hydrolyzed by addition of aqueous sodium hydroxide solution (other aqueous bases) and the product is extracted with toluene (other no-water miscible solvents or precipitated/crystallized by anti-solvent addition) and isolated by removing the solvent under reduced pressure at 60° C. (XXVIII) is purified by distillation under vacuum at 125-160° C.
This process has the advantage of giving 7-methoxy-5-methyl-1-benzothiophene (XXVII) in high yield and high purity without impurities according to Scheme 1 which are critical in regard of the quality of the final pharmaceutical ingredient (I) for clinical applications and cannot be easily purged in one of the following process steps towards (I).
This process has the advantage of giving 7-methoxy-5-methyl-1-benzothiophene (XXVII), using standard multipurpose equipment and safe reagents on industrial scale. The use of drastic reaction conditions like high temperatures >160° C. and unfavourable reagents like syrup-like polyphosphoric acid, which is not completely dissolved in the reaction mixture, is avoided. Very costly safety and engineering considerations on industrial scale are therefore avoided.

Step 5:

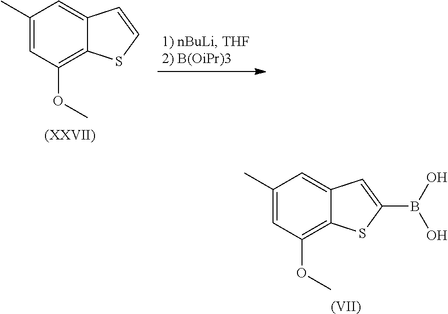

According to the first aspect of the present invention, the reaction of (XXVII) to benzothiophen-2-yl boronates of the formula (VIII) is done by borylation. (XXVII) is dissolved in an inert solvent such as THF and metallated by addition to a metal organic base such as n-butyl lithium solution in THF/hexane at −73 to −80° C. After stirring the reaction mass for 30 minutes triisopropyl borate is slowly added at −73 to −80° C. After a reaction time of 30 minutes, the mixture is hydrolyzed with aqueous potassium hydroxide solution at <10° C. and phases are separated at 20-30° C. Aqueous phase is washed with toluene and product is precipitated by addition of aqueous sulfuric acid solution at 0-5° C. (other acids). (XXVIII) is isolated by filtration and washed with water. The product is reslurried with a solvent such as cyclohexane at 40-45° C., isolated and dried at 40-45° C. at reduced pressure.
This process has the advantage of giving (7-methoxy-5-methyl-1-benzothiophen-2-yl)boronic acid (VIII) in high yield and high purity without impurities according to Scheme 1 which are critical in regard of the quality of the final pharmaceutical ingredient (I) for clinical applications and cannot be easily purged in one of the following process steps towards (I).

In addition to the method for preparing (VIII) from (XXVII) as described in European Patent Application No. 15180755.9, a second method for preparing (VIII) from (XXVII) is by dissolving (XXVII) in an inert solvent such as THF and metallating by addition to a metal organic base such as n-butyl lithium solution in THF/hexane at −55 to −80° C. After stirring the reaction mass for 30 minutes triisopropyl borate is added at −55 to −80° C.

After a reaction time of 30 minutes, the mixture is warmed up to −10° C. and hydrolyzed with aqueous potassium hydroxide solution at <30° C. and phases are separated at 20-30° C. Aqueous phase is washed with toluene and the mixture is acidified by addition of aqueous sulfuric acid solution at 20° C. 2-Propanol is added and the product is crystallized by distilling of the organic solvents at elevated temperature and reduced pressure. (XXVIII) is isolated by filtration and washed with water. The product is reslurried with a solvent such as cyclohexane at 40-45° C., isolated and dried at 40-45° C. at reduced pressure.

### Definitions

Solvates in the context of the invention are designated as those forms of the compounds according to the invention which form a complex in the solid or liquid state by stoichiometric coordination with solvent molecules.

Hydrates are a specific form of solvates, in which the coordination takes place with water. Hydrates are preferred solvates in the context of the present invention.

The compounds of this invention may, either by nature of asymmetric centers or by restricted rotation, be present in the form of isomers (enantiomers, diastereomers). Any isomer may be present in which the asymmetric center is in the (R)-, (S)-, or (R,S)-configuration.

All isomers, whether separated, pure, partially pure, or in racemic mixture, of the compounds of this invention are encompassed within the scope of this invention. The purification of said isomers and the separation of said isomeric mixtures may be accomplished by standard techniques known in the art. For example, diastereomeric mixtures can be separated into the individual isomers by chromatographic processes or crystallization, and racemates can be separated into the respective enantiomers either by chromatographic processes on chiral phases or by resolution.

In addition, all possible tautomeric forms of the compounds described above are included according to the present invention.

The present invention also encompasses all suitable isotopic variants of the compounds according to the invention. An isotopic variant of a compound according to the invention is understood to mean a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body. Due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. Such modifications of the compounds according to the invention may therefore in some cases also constitute a preferred embodiment of the present invention. Isotopic variants of the compounds according to the invention can be prepared by processes known to those skilled in the art, for example by the methods described below and the methods described in the working examples, by using corresponding isotopic modifications of the particular reagents and/or starting compounds therein.

Unless otherwise noted, suitable bases for the coupling reactions, where necessary, are in particular alkali carbonates, such as sodium, potassium or caesium carbonate, alkali phosphates, such as sodium or potassium phosphate, or alkali fluorides, such as potassium or caesium fluoride. Usually, these bases are employed as aqueous solutions. The reactions are carried out in organic solvents that are inert under the reaction conditions. Preferably, water-miscible organic solvents, such as 1,2-dimethoxyethane, tetrahydrofuran, 1,4-dioxane, acetonitrile, N,N-dimethylformamide (DMF) or dimethylsulfoxide (DMSO), are employed but other inert solvents, such as dichloromethane or toluene, may also be used.

Unless otherwise noted, condensing agents suitable for the process steps, where necessary, include, for example, carbodiimides such as N,N′-diethyl-, N,N′-dipropyl-, N,N′-diisopropyl-, N,N′-dicyclo-hexylcarbodiimide (DCC) or N-(3-dimethylaminopropyl)-N′-ethylcarbodiimide (EDC), phosgene derivatives such as N,N′-carbonyldiimidazole (CDI) or isobutyl chloroformate, α-chloroenamines such as 1-chloro-2-methyl-1-dimethylamino-1-propene, phosphorus compounds such as propane-phosphonic anhydride, diethyl cyanophosphonate, bis(2-oxo-3-oxazolidinyl)phosphoryl chloride, benzotriazol-1-yloxy-tris(dimethylamino)phosphonium hexafluorophosphate (BOP) or benzo-triazol-1-yloxy-tris(pyrrolidino)phosphonium hexafluorophosphate (PyBOP), and uronium compounds such as O-(benzotriazol-1-yl)-N,N,N′,N′-tetramethyluronium tetrafluoroborate (TBTU), 0-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), if appropriate in combination with further auxiliaries, such as 1-hydroxybenzotriazole (HOBt) or N-hydroxysuccinimide (HOSu), and/or bases such as alkali carbonates, for example sodium or potassium carbonate, or organic amine bases, such as triethylamine, N-methylpiperidine, N-methylmorpholine (NMM), N,N-diisopropylethylamine (DIPEA), pyridine or 4-N,N-dimethylaminopyridine (DMAP). Preference is given to using 0-(7-azabenzotriazol-1-yl)-N,N,N′,N′-tetramethyluronium hexafluorophosphate (HATU) or O-(benzotri-azol-1-yl)-N,N,N′,N′-tetramethyluronium tetrafluoroborate (TBTU) in combination with N,N-diisopropylethylamine (DIPEA) and optionally 1-hydroxybenzotriazole (HOBt).

Unless otherwise noted, acceptable inert solvents for process (where necessary) are, for example, ethers such as diethyl ether, tert-butyl methyl ether (MTBE), tetrahydrofuran (THF), 1,4-dioxane or 1,2-dimethoxyethane, hydrocarbons such as benzene, toluene, xylene, hexane or cyclohexane, halogenated hydrocarbons such as dichloromethane, trichloromethane, carbon tetrachloride, 1,2-dichloroethane, trichloroethylene or chlorobenzene, or other solvents such as acetone, acetonitrile, ethyl acetate (EtOAC), pyridine, dimethylsulfoxide (DMSO), N,N-dimethylformamide (DMF), N,N′-dimethylpropylene urea (DMPU) or N-methylpyrrolidinone (NMP). It is also possible to use mixtures of these solvents. Preference is given to using dichloromethane, tetrahydrofuran, N,N-dimethylformamide or mixtures thereof.

Method for Treatment:

The crystalline forms of the compound of formula (I), preferably crystalline form (III) according to the invention may have useful pharmacological properties and may be employed for the prevention and treatment of disorders in humans and animals. The forms of the compound of formula (I) according to the invention may open up a further treatment alternative and may therefore be an enrichment of pharmacy.

The crystalline forms of the compound of formula (I) according to the invention can be utilized to inhibit, block, reduce, decrease, etc., cell proliferation and/or cell division, and/or produce apoptosis.

This method comprises administering to a mammal in need thereof, including a human, an amount of a compound of general formula (I) of the present invention, which is effective to treat the disorder. Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypothalmic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

In some embodiments, the present invention further relates to a method for the treatment and/or prophylaxis of diseases, in particular the aforementioned diseases, using an effective amount of at least one of the forms of the compound of formula (I) according to the invention.

In some embodiments, the present invention further relates to a method for the treatment and/or prophylaxis of bladder cancer using an effective amount of at least one of the forms of the compound of formula (I) according to the invention.

In some embodiments, the present invention further relates to a method for the treatment and/or prophylaxis of head and neck cancer using an effective amount of at least one of the forms of the compound of formula (I) according to the invention.

In some embodiments, the present invention further relates to a method for the treatment and/or prophylaxis of lung cancer using an effective amount of at least one of the forms of the compound of formula (I) according to the invention.

The forms of the compound of formula (I) according to the invention can be used alone or in combination with other active substances if necessary. The present invention further relates to medicinal products containing at least one of the forms of the compound of formula (I) according to the invention and one or more further active substances, in particular for the treatment and/or prophylaxis of the aforementioned diseases. As suitable other active substances the following can be mentioned:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (1231), iomeprol, ipilimumab, irinotecan, Itraconazole, ixabepilone, ixazomib, lanreotide, lansoprazole, lapatinib, Iasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, and zorubicin.

Pharmaceutical Compositions:

It is possible for the crystalline form of the compound of formula (I) according to the present invention to have systemic and/or local activity. For this purpose, it can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the crystalline form of the compound of formula (I) according to the present invention to be administered in suitable administration forms.

For oral administration, it is possible to formulate the crystalline form of the compound of formula (I) according to the present invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compound according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The crystalline form of the compound of formula (I) can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat), e solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least the crystalline form of the compound of formula (I) according to the present invention, conventionally together with one or more pharmaceutically suitable excipient(s), and to their use according to the present invention.

Dosage of the Pharmaceutical Compositions of the Present Invention:

Based upon laboratory techniques known to evaluate compounds useful for the treatment of disorders, by pharmacological assays for the determination of treatment of the conditions identified above in mammals, and by comparison of these results with the results of known medicaments that are used to treat these conditions, the effective dosage of the compound of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

The weight data in the tests and examples which follow are, unless stated otherwise, percentages by weight; parts are parts by weight. Solvent ratios, dilution ratios and concentration data of liquid/liquid solutions are based on each case on the volume.

### EXAMPLES

### Abbreviations and Acronyms

Ac acetyl
Ac₂O acetic anhydride
AcOH acetic acid
aq. aqueous (solution)
Boc tert-butoxycarbonyl
br. broad (¹H-NMR signal)
Bu butyl
cat. catalytic
conc. concentrated
d doublet (¹H-NMR signal)
DBDMH 1,3-dibromo-5,5-dimethylhydantoin
DCI direct chemical ionization (MS)
DCM dichloromethane
Dess-Martin periodinane 1,1,1-triacetoxy-1,1-dihydro-1,2-benziodoxol-3(H)-one
DIPEA N,N-diisopropylethylamine
DMF N,N-dimethylformamide
DMSO dimethylsulfoxide
EI electron impact ionization (MS)
eq. equivalent(s)
ESI electro-spray ionization (MS)
Et ethyl
EtOAc ethyl acetate
GC-MS gas chromatography-coupled mass spectroscopy
h hour(s)
Hal halogen
¹H-NMR proton nuclear magnetic resonance spectroscopy
HPLC high performance liquid chromatography
iPr isopropyl
LC-MS liquid chromatography-coupled mass spectroscopy
Me methyl
MeOH methanol
min minute(s)
MS mass spectroscopy
m/z mass-to-charge ratio (MS)
NBS N-bromosuccinimide
n-Bu n-butyl
NCS N-chlorosuccinimide
of th. of theory (chemical yield)
Pd/C palladium on charcoal
PdCl₂(dppf) [1,1′-bis(diphenylphosphino)ferrocene]dichloropalladium(II)
Pd(dba)₂ bis(dibenzylideneacetone)palladium
Ph phenyl
PPA polyphosphoric acid
q quartet (¹H-NMR signal)
quant. quantitative (yield)
rac racemic
Rr TLC retention factor
RP reverse phase (HPLC)
rt room temperature
Rₜ retention time (HPLC)
s singlet (¹H-NMR signal)
sat. saturated (solution)
t triplet (¹H-NMR signal)
TBAF tetra-n-butylammonium fluoride
TBDMS tert-butyldimethylsilyl
TBTU N-[(1H-benzotriazol-1-yloxy)(dimethylamino)methylene]-N-methyl-methanaminium tetrafluoroborate
tBu tert-butyl
tert tertiary
TFA trifluoroacetic acid
THF tetrahydrofuran
TLC thin layer chromatography

Methods:

DSC/TG

DSC thermograms were recorded using Differential Scanning Calorimeters (model DSC7, Pyris-1 or Diamond) from Perkin-Elmer. The measurements were performed with a heating rate of 20 Kmin⁻¹, eventually 2 Kmin⁻¹ using non-gastight aluminium pans. Flow gas was nitrogen. There was no sample preparation.

TGA thermograms were recorded using thermobalances (model TGA7 and Pyris 1) from Perkin-Elmer.

The measurements were performed with a heating rate of 10 Kmin⁻¹ using open platinum pans. Flow gas was nitrogen. There was no sample preparation.

XRPD

X-Ray diffraction patterns were recorded at room temperature using XRD-diffractometers X'Pert PRO (PANalytical) and STOE STADI-P (radiation Cu K alpha 1, wavelength 1.5406 Å). There was no sample preparation. All X-Ray reflections are quoted as °2θ (theta) values (peak maxima) with a resolution of ±0.2°.

Raman

Raman spectra were recorded at room temperature using FT-Raman-spectrophotometers (model RFS 100 and MultiRam, Wavenumber range: 3500-100 cm⁻¹) from Bruker. Resolution was 2 cm⁻¹. Number of scans: 64. Measurements were performed in glass vials or aluminium discs. There was no sample preparation.

IR

IR spectra were recorded at room temperature using an IR Spectrometer Bruker Tensor 37 with HATR-device in a wavenumber range of 4000 to 550 cm-1. Resolution was 2 cm-1. Number of scans: 64. There was no sample preparation.

HPLC (Method 1):

System: High performance liquid chromatograph equipped with gradient pumps, UV detector & attached with data recorder and integrator software; column: Waters XBridge Phenyl (150 mm*2.1 mm, 3.5 m); flow: 0.5 mL/min; column temperature: 25° C.; detection 226 nm, run time: 30 min; mobile phase A: 385 mg CH₃COONH₄ in 1 L deionized water, pH 8.5 adjusted with NH₄OH 25% (ca. 70 μl); mobile phase B: acetonitrile;

| Gradient: | time (min) | B (%) |
|---|---|---|
| | 0.00 | 5.00 |
| | 20.00 | 45.0 |
| | 25.00 | 80.0 |
| | 30.00 | 80.0 |

HPLC (Method 2):

System: High performance liquid chromatograph equipped with gradient pumps, UV detector & attached with data recorder and integrator software; column: Waters XBridge Phenyl (150 mm*2.1 mm, 3.5 μm); flow: 0.4 mL/min; column temperature: 25° C.; detection 226 nm, run time: 30 min; mobile phase A: 385 mg CH₃COONH₄ in 1 L deionized water, pH 8.5 adjusted with NH₄OH 25% (ca. 70 μl); mobile phase B: acetonitrile;

| Gradient: | time (min) | B (%) |
|---|---|---|
| | 0.00 | 5.00 |
| | 20.00 | 45.0 |
| | 25.00 | 80.0 |
| | 30.00 | 80.0 |

HPLC (Method 3):

System: High performance liquid chromatograph equipped with gradient pumps, UV detector & attached with data recorder and integrator software; column: Waters XBridge Phenyl (150 mm*2.1 mm, 3.5 μm); flow: 0.4 mL/min; column temperature: 25° C.; detection 226 nm, run time: 30 min; mobile phase A: 770 mg CH₃COONH₄ in 0.7 L deionized water, pH 8.5 adjusted with NH₄OH 25% (ca. 200 μl); mobile phase B: acetonitrile;

| Gradient: | time (min) | B (%) |
|---|---|---|
| | 0.00 | 5.00 |
| | 20.00 | 45.0 |
| | 25.00 | 80.0 |
| | 30.00 | 80.0 |

HPLC (Method 4):

System: High performance liquid chromatography system, equipped with a degasser, delay volume (dwell volume) of approx. 850 μL, UV-VIS Detector and chromatography data system. Stationary phase: Meteoric Core C18 (150 mm length, 3.0 mm ID, 2.7 μm particle size); mobile phase A: 1.15 g NH₄H2PO₄+155 μL H₃PO₄ 85%/1 L water (pH 3.0); mobile phase B: acetonitrile/methanol 52:48 v/v; UV detection at 226 nm; oven temperature: 65° C., injection volume: 6 μl; linear gradient:

| Gradient: | time (min) | flow (mL/min) | A (%) | B (%) |
|---|---|---|---|---|
| | 0 | 0.90 | 80.0 | 20.0 |
| | 8.5 | 0.90 | 62.0 | 38.0 |
| | 13.0 | 0.90 | 50.5 | 49.5 |
| | 21.0 | 1.20 | 30.2 | 69.8 |
| | 25.0 | 1.20 | 20.0 | 80.0 |
| | 35.0 | 1.20 | 20.0 | 80.0 |

HPLC (Method 5):

System: High performance liquid chromatograph equipped with gradient pumps, UV detector & attached with data recorder and integrator software; column: Kromasil C18 (250 mm*4.6 mm, 5 μm); flow: 0.4 mL/min; column temperature: 25° C.; detection 226 nm, run time: 30 min; mobile phase A: deionized water with 0.1% H₃PO₄, mobile phase B: acetonitrile;

| Gradient: | time (min) | B (%) |
|---|---|---|
| | 0.00 | 10 |
| | 20.00 | 90 |
| | 30.00 | 90 |
| | 31.00 | 10 |

Residual Solvents (Determined by GC Method 1):

System: Headspace injector, gas chromatograph with splitter, autosampler, 2 flame ionisation detector (FID) and data analysis system.

Residual Elements: ICP-MS

### Example 1: tert-butyl 1H-pyrrol-1-ylcarbamate (XI)

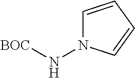 [SMILES: CC(C)(C)OC(=O)Nn1cccc1]

A stirred reaction vessel was initially charged with 1045 kg of dioxane, 350 kg of tert-butyl hydrazinecarboxylate and 420 kg 2,5-dimethoxytetrahydrofuran, and then 21.4 kg of pyridine hydrochloride and 343 kg of pyridine were added. Transfer lines were rinsed with a total amount of 40 L of dioxane. The reaction mixture was heated to 102±3° C. for 7 h and 1620 L of solvents were distilled of. The batch was cooled down to 25±5° C. and 1750 kg of water were charged into the reactor over 1 h keeping the temperature at 22±3° C. The mixture was stirred for at least 30 min, then 270 kg of di-nbutyl ether were added and the temperature was adjusted to 10±3° C. The suspension was stirred for 2 h and then isolated on a centrifuge in two portions. Each portion was washed with a mixture of 67 kg di-nbutyl ether and 60 kg heptanes, followed by 175 L of heptanes. The product was dried at 60° C. and 288 kg of tert-butyl 1H-pyrrol-1-ylcarbamate (XI) were obtained in 60% of theoretical yield.

HPLC (Method 2): purity 98.66% (Rt=15.47 min) tert-butyl 1H-pyrrol-1-ylcarbamate

### Example 2: tert-butyl (2-cyano-1H-pyrrol-1-yl)carbamate (XII)

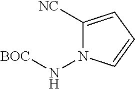 [SMILES: CC(C)(C)OC(=O)Nn1cccc1C#N]

190 kg of tert-butyl 1H-pyrrol-1-ylcarbamate (XII) and 678 kg of anhydrous DMF were stirred at 22±3° C. in a reaction vessel until the product was dissolved. The batch was cooled to 0±3° C. and 163 kg of chlorosulfonyl isocyanate were slowly added over a minimum time period of 2.5 h keeping the mixture at this temperature. The mixture was stirred for one additional hour. In a second stirred reaction vessel a solution of 272 kg ammonium hydrogen carbonate in 2660 kg of water was provided and the reaction mixture was transferred onto this solution by maintaining a temperature below 25° C. The first reactor and the transfer line were rinsed with 20 L of DMF followed by 20 L of water.

The mixture was kept stirring at 22±3° C. for 2 h, the crude product was isolated on a centrifuge in two portions, and each portion was washed twice with 760 L of water.

The crude product was dissolved in 452 kg of methanol at 37±3° C. in a stirred reaction vessel. 1197 kg of water were provided in a second reactor at 22±3° C. and the methanolic solution in was dosed into the water within 1 h keeping the temperature at 22±3° C. The first reactor and the transfer line were rinsed with 20 L of water and the mixture was stirred for 2 h. The product was isolated on a centrifuge in two portions, and each portion was washed with 143 L of a mixture of water and methanol of 4:1 volumes. 173 kg tert-butyl (2-cyano-1H-pyrrol-1-yl)carbamate (XII) were obtained in 80% of theoretical yield after drying at 50° C.

HPLC (method 3): purity 98.7% (Rt=17.28 min) tert-butyl (2-cyano-1H-pyrrol-1-yl)carbamate

### Example 3: tert-butyl (4-bromo-2-cyano-1H-pyrrol-1-yl)carbamate (IX)

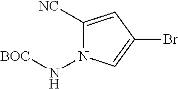 [SMILES: CC(C)(C)OC(=O)Nn1cc(Br)cc1C#N]

180 kg of tert-butyl (2-cyano-1H-pyrrol-1-yl)carbamate (XII) and 508 kg of DMF were stirred in a reaction vessel and 666 kg methyl tert-butyl ether were added. The batch was cooled to 2±3° C. and 171 kg of N-bromosuccinimide were added in 15 portions within 2.5 h at this temperature.

The mixture was hydrolysed by addition of a solution of 13.1 kg sodium sulphite in 740 L of water within 40-50 min, keeping the temperature below 25° C. The batch was stirred at 22±3° C. for 30-60 min, and then the lower aqueous phase was discarded. The organic phase was washed twice with 360 L of water and approximately 540 L of solvent were removed by distillation at 45±5° C. under reduced pressure. The residual mixture was diluted with 310 kg of methyl tetrahydrofurane and approximately 360 L of solvent were removed by distillation at 45±5° C. under reduced pressure.

310 kg of methyl tetrahydrofurane were added and 733 kg of a solution of tert-butyl (4-bromo-2-cyano-1H-pyrrol-1-yl)carbamate (IX) was obtained and converted in the next chemical step without further purification.

### Example 4: tert-butyl 12-cyano-4-(hydroxymethyl)-1H-pyrrol-1-ylcarbamate (IV)

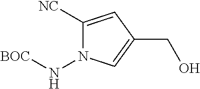 [SMILES: CC(C)(C)OC(=O)Nn1cc(CO)cc1C#N]

The solution of tert-butyl (4-bromo-2-cyano-1H-pyrrol-1-yl)carbamate (IX) in methyl tetrahydrofurane from previous stage, corresponding to conversion of 140 kg of tert-butyl (2-cyano-1H-pyrrol-1-yl)carbamate (XII), was stirred in a first reaction vessel, diluted with 950 kg of methyl tetrahydrofurane and cooled to −40±3° C. 88.6 kg of a 35% solution of methyl magnesium bromide in methyl tetrahydrofurane was added, keeping the temperature at −40±3° C., followed by 10 L methyl tetrahydrofurane. The mixture was stirred for 30 min at −43±6° C. and then 123 kg of a solution of 23% butyl lithium in hexane was added, keeping the temperature at −43±6° C., followed by 10 L methyl tetrahydrofurane. The mixture was stirred for 45-60 min at this temperature.

Treatment of paraformaldehyde: 81 kg of paraformaldehyde were stirred in 243 L of methyl tetrahydrofurane at ambient temperature, isolated by filtration and approximately 88 kg of solvent-wet paraformaldehyde were obtained.

A second stirred reaction vessel was charged with 546 kg of methyl tetrahydrofurane and the solvent-wet paraformaldehyde. The temperature was adjusted to 24±3° C. and the cold reaction mixture from the first reaction vessel was transferred via an insulated pipe into the paraformaldehyde slurry within 2 h keeping the batch temperature at 24±3° C. The first reactor and the transfer line was rinsed with 40 L of methyl tetrahydrofurane and the batch was stirred for 1.5 h at 24±3° C.

In a third stirred reaction vessel a solution of 193 kg ammonium chloride in 840 L of water was prepared at 12±3° C. and the reaction mixture was dosed into this solution, followed by 40 L of methyl tetrahydrofurane keeping the temperature at 12±3° C. A solution of 155 kg citric acid in 280 L of water was added and the mixture was stirred at 12±3° C. for at least 15 min. The lower aqueous layer was discarded and the organic phase was washed with 280 kg of 15% aqueous sodium chloride solution. The batch was concentrated by distillation under reduced pressure keeping the batch temperature at 45±5° C. until a residual volume of around 560 L.

The starting of the procedures was repeated for another time in analogues fashion and the two batches were combined in a stirred reaction vessel.

Total Batch Size Now Corresponds to a Total Amount of 280 kg tert-butyl (2-cyano-1H-pyrrol-1-yl)carbamate (XII) being Converted Through the Sequence of Bromination and Metalation and Addition to Paraformaldehyde.

The mixture resulting from two batches was concentrated to a residual volume of about 600 L by distillation under reduced pressure keeping the batch temperature at 45±5° C., and then 1400 L n-heptane were added. About 1400 L of solvent were removed by distillation under reduced pressure keeping the batch temperature at 45±5° C. The distillation was stopped and the batch was cooled to 25±5° C. Then 1400 L n-heptane were added and about 1400 L of solvent were removed by distillation under reduced pressure keeping the batch temperature at 45±5° C. 140 L of ethyl acetate were added at 50±3° C. and the batch was kept at this temperature for 20-30 min, cooled to 2±3° C. and stirred for 1 h. The product was isolated on a centrifuge and washed twice with 140 L of a mixture of 2 volumes n-heptane and 1 volume of ethyl acetate. The product was dried at 40° C. and 184 kg (57% of theoretical yield) were obtained.

HPLC (Method 2): purity 97.8% (Rt=13.83 min) tert-butyl [2-cyano-4-(hydroxymethyl)-1H-pyrrol-1-yl]carbamate

### Example 5: 6-(methoxymethyl)pyrrolo[2,1-f][1,2,4]triazin-4-amine (V)

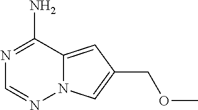

A stirred reaction vessel was charged with 905 kg of 13.6% HCl in dioxane solution and the temperature was adjusted to 25+/−5° C. 200 kg of tert-butyl [2-cyano-4-(hydroxymethyl)-1H-pyrrol-1-yl]carbamate (IV) were added in 5 portions within 140 min, keeping the temperature below 30° C. The mixture was stirred for 6 h at 22+/−3° C. In another stirred reaction vessel a mixture of 948 kg MeOH and 380 kg of a solution of 30% sodium methylate in methanol was provided and the reaction mixture was dosed into this solution, keeping the temperature at 15-30° C. The reactor and the transfer line was rinsed with 102 kg of dioxane and the reaction mixture was stirred at 22+/−3° C. for 1 h. 526 kg of formamidine acetate were added and the batch was heated to 63+/−3° C. and kept at this temperature for 18.5 h.

A solution of 1163 kg K₃PO₄ in 2597 kg of water was added and the reaction mixture was kept at 63+/−3° C. for 2 h.

The mixture was concentrated by distillation at a maximum internal temperature of 40° C. at 500-100 mbar, until no more distillate was collected (approx. 2450 L of distillate). The reaction mixture was heated to 45+/−3° C. and 1390 kg of iso-propyl acetate were added. The biphasic mixture was stirred for 30 min at this temperature, then the layers were separated and the aqueous layer was again extracted with 1390 kg of iso-propyl acetate at 45+/−3° C. The organic phases were combined and passed through a filter at 45+/−3° C. into a stirred reaction vessel in order to remove insoluble matter.

The solution was concentrated at a maximum internal temperature of 45° C. at <300 mbar to a residual volume of approximately 600-800 L.

The mixture was heated to reflux at 95° C., kept at this temperature for at least 30 min until a solution was obtained, and then slowly cooled down to 0+/−3° C. and kept at this temperature for 4 h. Then the product was isolated by filtration. The filter cake was washed with 176 kg of iso-propyl acetate and dried in an oven at 50° C. 102.5 kg of (V) was obtained as a solid in 68% yield.

HPLC (Method 5): purity 99.7% (6-(methoxymethyl)pyrrolo[2,1-f][1,2,4]triazin-4-amine) (Rt=4.6 min), 0.09% (XXIV) at RRT 1.55, 0.05% (XXV)

Assay for use: 99.0% (against external standard)

Recrystallization Process

A stirred reaction vessel was charged with 510 g (V) (purity by hplc: 89.7 area % and 85.2% assay for use), 1020 mL isopropyl acetate and 331 mL ethanol and heated to reflux for 1 h. The mixture was cooled to 0° C. within 3 h and stirred at 0° C. for additional 2 h. Purified (V) was isolated by filtration and washed three times with 510 mL cold isopropyl acetate. (V) was dried at 30° C. at reduced pressure and 331 g (V) (66.7% yield) were obtained HPLC (method 1: purity 99.6% (6-(methoxymethyl)pyrrolo[2,1-f][1,2,4]triazin-4-amine) Assay for use: 100.6% (against external standard)

### Example 7: 4-{[4-amino-5-bromo-6-(methoxymethyl)pyrrolo[2,1-f][1,2,4]triazin-7-yl]methyl}piperazin-2-one (VII)

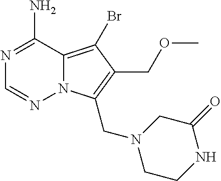 [SMILES: COCc1c(Br)c2c(N)ncnn2c1CN1CCNC(=O)C1]

Method 1: pH 7.2:

A stirred reaction vessel was charged with 93.8 kg 6-(methoxymethyl)pyrrolo[2,1-f][1,2,4]triazin-4-amine (V), 78.8 kg piperazin-2-one and 726 kg methanol, heated to 22±3° C. and stirred for at least 30 min at this temperature. The mixture was passed through a filter in order to remove insoluble matter and the filtrate was collected in another stirred reaction vessel. The first vessel and the filter were rinsed with 178 kg of methanol. The temperature was adjusted to 22±3° C. and 188 kg acetic acid were added at this temperature, followed by 20 kg of methanol. 17.4 kg paraformaldehyde were added and the batch was heated to reflux for 24 h. The mixture was cooled down to 25±5° C. and after completion of the reaction (compound (V)≤6.0% according to HPLC) the reaction mixture was neutralized to pH 7.2±0.2 keeping the batch temperature at 25±5° C. by addition of 146 kg 50% aqueous sodium hydroxide solution, followed by 20 kg of methanol.

The mixture was cooled to −5±3° C. and 103.2 kg N-bromosuccinimide were charged into the reactor in small portions over a period of not less than 2 h keeping the batch temperature at −5±3° C. After the end of the addition, the mixture is stirred for at least 15 min, and then was heated to reflux for 1 h, again cooled to 22±3° C. within 2 h and hold at this temperature for an additional hour. The crude product was isolated by centrifugation, mother liqueur can be recirculated through the centrifuge to finalize isolation of all material. The isolated crude product was washed twice with 148 kg of methanol.

The crude reaction product was transferred into a stirred reaction vessel together with 741 kg of methanol, heated to 25±5° C. and stirred for at least 10 min at this temperature. The mixture was heated to reflux for 3 h to 3.5 h, again cooled to 10±3° C. within 3 h and hold at this temperature for an additional hour. The purified product was isolated by centrifugation and the filter cake was washed twice with 148 kg of methanol.

The purified product was transferred into a stirred reaction vessel together with 741 kg of methanol, heated to 25±5° C. and stirred for at least 10 min at this temperature. The mixture was heated to reflux for 3 h to 3.5 h, again cooled to 10±3° C. within 3 h and hold at this temperature for an additional hour. The pure product was isolated by centrifugation and the filter cake was washed twice with a mixture of 74 kg methanol and 94 kg water.

The product was dried in thin layers on trays (ca. 2.9 kg/m²) in a tray dryer by air ventilation at ambient temperature and 94.9 kg of 4-{[4-amino-5-bromo-6-(methoxymethyl)pyrrolo[2,1-f][1,2,4]triazin-7-yl]methyl}piperazin-2-one (VII) (45% yield) were obtained as a hydrate, containing approximately 1 mol of water per mol of (VII).

HPLC (Method 3): purity 99.8% (4-{[4-amino-5-bromo-6-(methoxymethyl)pyrrolo[2,1-f][1,2,4]triazin-7-yl]methyl}piperazin-2-one) (RT=10.8 min), relevant by-products: (XXI) at RRT (relative retention time) of 1.05:0.09%; (XX) at RRT 1.38:0.07%; (VI) at RRT 0.74: not detected

Assay for use: 96.1% (against external standard)

TGA: 5.0% loss of weight

Method 2:

A stirred reaction vessel was charged with 140.0 kg 6-(methoxymethyl)pyrrolo[2,1-f][1,2,4]triazin-4-amine (V), 117.6 kg piperazin-2-one and 1106 kg methanol, and the mixture was stirred for 1 h at 22±3° C. The mixture was passed through a filter in order to remove insoluble matter and the filtrate was collected in another stirred reaction vessel. The first vessel and the filter were rinsed with 266 kg of methanol. 280 kg of acetic acid were added at 22±3° C. temperature, followed by 20 L of methanol. 26 kg Paraformaldehyde were added and the batch was heated to reflux for 24 h. The mixture was cooled down to 25±5° C. and after completion of the reaction (compound (V)≤6.0% according to hplc) the reaction mixture was neutralized to pH 6.0±0.2 keeping the batch temperature at <25° C. by addition of 84 kg 50% aqueous sodium hydroxide solution, followed by 20 L of methanol. The mixture is stirred at 22±3° C. for at least 15 min and then cooled to −5±3° C.

In a separate stirred vessel 154 kg of N-bromo succinimide were dissolved in 934 kg of acetonitrile at 20° C. The NBS solution was transferred into the reaction mixture by keeping the temperature at −5±3° C. (dosing time >4 h). Residues of NBS solution were flushed with 50 L of ACN. The mixture was then stirred for 30 to 40 min at this temperature and then was heated to reflux for 1 h, again cooled to 22±3° C. within 1 h and hold at this temperature for an additional hour. The crude product was isolated by filtration. The filter cake was washed twice with 221 kg of methanol.

The crude reaction product was transferred into a stirred reaction vessel together with 1106 kg of methanol, heated to 25±5° C. and stirred for at least 10 min at this temperature. The mixture was heated to reflux for 3 h to 3.5 h, again cooled to 10±3° C. within 3 h and hold at this temperature for an additional hour. The purified product was isolated by filtration and the filter cake was washed twice with 221 kg of methanol.

The purified product was transferred into a stirred reaction vessel together with 995 kg of methanol and 140 kg of water heated to 25±5° C. and stirred for at least 10 min at this temperature. The mixture was heated to reflux for 3 h to 3.5 h, cooled to 10±3° C. within 3 h and hold at this temperature for an additional hour. The pure product was isolated by filtration and the filter cake was washed twice with a mixture of 111 kg methanol and 140 kg of water.

The product was dried in thin layers on trays (ca. 2.9 kg/m²) in a tray dryer by air ventilation at ambient temperature and 208 kg of 4-{[4-amino-5-bromo-6-(methoxymethyl)pyrrolo[2,1-f][1,2,4]triazin-7-yl]methyl}piperazin-2-one (72% yield) were obtained as a hydrate, containing approximately 1 mol of water per mol of (VII).

HPLC (Method 3): Lot 14 purity 99.5% (4-{[4-amino-5-bromo-6-(methoxymethyl)pyrrolo[2,1-f][1,2,4]triazin-7-yl]methyl}piperazin-2-one) (RT=11.1 min), relevant by-products: (XXI) at RRT (relative retention time) of 1.05:0.15%; (XX) at RRT 1.38 min: 0.16%; (VI) at RRT 0.74: <0.05%

Assay for use: 95.1% (against external standard, a hydrate contains ca. 1 mol/5% of water)

### Example 8: 4-{[4-amino-6-(methoxymethyl)-5-(7-methoxy-5-methyl-1-benzothiophen-2-yl)pyrrolo[2,1-f][1,2,4]triazin-7-yl]methyl}piperazin-2-one (I)

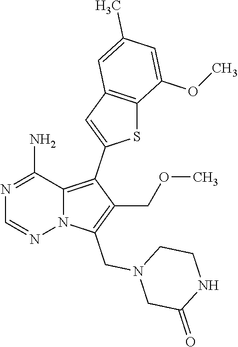 [SMILES: COCc1c(-c2cc3cc(C)cc(OC)c3s2)c2c(N)ncnn2c1CN1CCNC(=O)C1]

27.1 kg 4-{[4-amino-5-bromo-6-(methoxymethyl)pyrrolo[2,1-f][1,2,4]triazin-7-yl]methyl}piperazin-2-one (VII) were placed in 425 kg THF in a stirred reaction vessel at 10° C. jacket temperature. 74 kg of water, 22.1 kg of (7-methoxy-5-methyl-1-benzothiophen-2-yl)boronic acid (VIII) and 20.5 kg of potassium carbonate were added and the reaction mixture was inerted by reducing internal pressure to 200 mbar and refilling to ambient pressure with argon gas. 0.47 kg [1,1′-Bis(di-tert-butylphosphino)ferrocene]dichloropalladium(II) were added and the reactor was inerted by reducing internal pressure to 200 mbar and refilling to ambient pressure with argon gas. The reaction mixture was heated to reflux (approx. 64° C. internal temperature) within 90 min and stirred for additional 2 h at this temperature. A solution of 24.1 kg acetyl cysteine in 274 kg water was added at 55° C. to reflux temperature. The reaction mixture was stirred at this temperature for 2 h, then 242 kg of ethyl acetate were added at 55° C. to reflux temperature. The jacket temperature was set to 50° C.

After stirring for 60 min, the reaction mixture was concentrated by distilling off 366 kg of solvent from the reaction mixture at 48° C. to 50° C. and 500 mbar. Additional 239 kg of ethyl acetate were added and the reaction mixture was concentrated by distilling off 147 kg of solvent from the reaction mixture at 46° C. to 50° C. and 500 mbar. After the distillation is finished, the jacket temperature was adjusted to 75° C. and the mixture was stirred for 60 min. The mixture was cooled to 20° C. internal temperature within 2 h and stirred for additional 2 h. The product was isolated by filtration and washed with a mixture of 196 kg ethanol and 22 kg water.

The product is placed in to a stirred reaction vessel in a mixture of 456 kg THF and 91 kg water and heated to 65° C. until a solution was obtained. The jacket temperature was set to 50° C. and the reaction mixture was concentrated by distillation at 500 to 200 mbar, until no more distillate is collected (approximately 469 kg of distillate). 238 kg of ethanol were added and the reaction mixture was concentrated by distilling off 46 kg of solvent from the reaction mixture at 140 mbar.

The jacket temperature was set to 80° C., the mixture was stirred for 3 h and was then cooled to 15° C. internal temperature within 3 h. The mixture was stirred for 1 h and the product was isolated on a centrifuge and washed with a mixture of 298 kg ethanol and 39 kg water. The product was dried at 45° C. and 30 mbar and 28.3 kg of (I) were obtained.

This procedure was repeated for producing a second batch of (I) by converting 27.4 kg 4-{[4-amino-5-bromo-6-(methoxymethyl)pyrrolo[2,1-f][1,2,4]triazin-7-yl]methyl}piperazin-2-one (VII) and 22.1 kg of (7-methoxy-5-methyl-1-benzothiophen-2-yl)boronic acid (VIII) in analogues fashion following method 1 and 28.5 kg of (I) were obtained.

Purification:

28.3 kg of (I) from the first batch of (I) were placed in a stirred reaction vessel in a mixture of 413 kg THF and 81 kg water and heated to reflux at 65° C. until a solution was obtained. Then the jacket temperature was set to 60° C. and the mixture was passed through a heated particle filter (60° C.) into another stirred reaction vessel.

28.5 kg of (I) from the second batch of (I) were placed in a stirred reaction vessel in a mixture of 410 kg THE and 81 kg water and heated to reflux at 65° C. until a solution was obtained. Then the jacket temperature was set to 60° C. and the mixture was passed through a heated particle filter (60° C.) into the stirred reaction vessel already containing the first portion of the solution of (I).

The jacket temperature was set to 50° C. and the reaction mixture was concentrated by distillation at 200 mbar, until no more distillate was collected (approximately 798 kg of distillate). 429 kg of ethanol were added and the reaction mixture was concentrated by distilling off 90 kg of solvent from the reaction mixture at 140 mbar.

The jacket temperature was set to 85° C., the mixture was stirred for 90 min and was then cooled to 15° C. internal temperature within 3 h. The mixture was stirred for 1 h and the product was isolated on a filter dryer and washed with a mixture of 354 kg ethanol and 45 kg water. Finally the product was dried at 45° C. and 30 mbar, yielding 53.6 kg of (I) in 78% of theoretical yield.

HPLC (Method 4):

purity: 99.8% (Rt=11.6 min.), relevant by-products: (VI) at RRT (relative retention time) of 0.16: n.d.; (XVIII) at RRT 1.02 min: 0.09%; (XV) at RRT 0.77: n.d. (XVI) at RRT 0.98: n.d.

Assay for use: 97.8% (against external standard)

Residual solvents (determined by GC method 1): 0.4% tetrahydrofuran

Residual elements (determined by ICP-MS): 0.9 mg/kg palladium

### Example 9: 4-{[4-amino-6-(methoxymethyl)-5-(7-methoxy-5-methyl-1-benzothiophen-2-yl)pyrrolo[2,1-f][1,2,4]triazin-7-yl]methyl}piperazin-2-one hydrochloride hydrate (III) monohydrate [A]

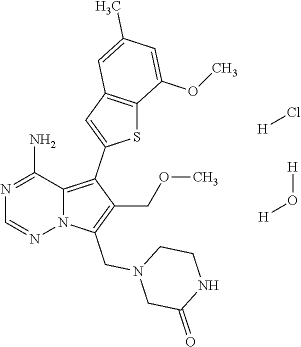 [SMILES: COCc1c(-c2cc3cc(C)cc(OC)c3s2)c2c(N)ncnn2c1CN1CCNC(=O)C1 ; Cl ; O]

Method 1:

16.1 kg of 4-{[4-amino-6-(methoxymethyl)-5-(7-methoxy-5-methyl-1-benzothiophen-2-yl)pyrrolo[2,1-f][1,2,4]triazin-7-yl]methyl}piperazin-2-one (I) were suspended in a mixture of 106 kg ethanol and 13 kg water by stirring in a reaction vessel. The mixture was heated to 50±5° C. and stirred for 1 h. 5.4±0.2 kg of a solution of 25% HCl in water was added, followed by 10 kg of ethanol and the batch was stirred for 1 h at this temperature. The mixture is cooled to 0±3° C. within 2.5 h and stirred at 0±3° C. for 1 h. The product is isolated by filtration and the filter is washed with 48 kg ethanol via the cold reaction vessel. The crude product is further processed without drying.

The process is carried out four times, thus converting a total of 64.4 kg. The four crude products were combined in the next process step.

2.5 kg 10% aqueous HCl solution were diluted by 193 kg of water in a stirred reaction vessel and the 4 crude products were suspended in this mixture. The batch was heated to 75±3° C., stirred at this temperature for 1 h and then cooled to 20±3° C. within 3 h. The product is isolated on a filter dryer and residual product is rinsed from the reaction vessel into the filter by circulating the filtrate via the reaction vessel. The product is dried at 30 mbar and 50° C. jacket temperature and 63.7 kg (89% yield) of 4-{[4-amino-6-(methoxymethyl)-5-(7-methoxy-5-methyl-1-benzothiophen-2-yl)pyrrolo[2,1-f][1,2,4]triazin-7-yl]methyl}piperazin-2-one hydrochloride hydrate (III) are obtained in the desired monohydrate [A] solid state form.

HPLC (Method 4):

purity: 99.7% (Rt=11.9 min.), relevant by-products: (VI) at RRT (relative retention time) of 0.16: n.d. (XVIII) at RRT 1.02 min: n.d.; (XV) at RRT 0.77:0.07%; (XVI) at RRT 0.98:0.160%

Assay for use: 98.2% (against external standard, based on monochloride, monohydrate)

Residual solvents (determined by GC method 1): 0.3% ethanol

Residual elements (determined by ICP-MS): 1 mg/kg palladium

Water content (Karl Fischer coloumetric): 3.5%

Ion chromatography: 6.4% chloride

Method 2:

51.1 kg to of 4-{[4-amino-6-(methoxymethyl)-5-(7-methoxy-5-methyl-1-benzothiophen-2-yl)pyrrolo[2,1-f][1,2,4]triazin-7-yl]methyl}piperazin-2-one (I) are suspended in a mixture of 336 kg ethanol and 41 kg water in a stirred reaction vessel. The mixture is heated to 50° C. and 17.3 kg) of a solution of 25% HCl in water, followed by 32 kg of ethanol is added. 0.48 kg 4-{[4-amino-6-(methoxymethyl)-5-(7-methoxy-5-methyl-1-benzothiophen-2-yl)pyrrolo[2,1-f][1,2,4]triazin-7-yl]methyl}piperazin-2-one hydrochloride hydrate (III) in the form of monohydrate [A] are added as seed crystals (e.g. prepared according to method 1 or method 2 and additionally milled by jet mill to below 20 μm particle size) and the reaction mixture is stirred for 1 h at 50° C.

The mixture is cooled to 0° C. within 2.5 h and stirred at 0° C. for 1 h. The product is isolated by filtration on a filter dryer and the filter is washed with 107 kg of cold ethanol. Then the filtration is stopped.

A mixture of 2.0 kg of a solution of 10% HCl in water is diluted with 154 kg of water and heated to 30° C. in the stirred reaction vessel and piped into the filter dryer. The filter cake is suspended on the filter dryer at 32° C. for 60 min by stirring and then filtrated.

The product is dried at 50° C. jacket temperature and 30 mbar until a product temperature of 45° C. is achieved. Then drying is continued for 2 h. 54.0 kg (95% yield) of 4-{[4-amino-6-(methoxymethyl)-5-(7-methoxy-5-methyl-1-benzothiophen-2-yl)pyrrolo[2,1-f][1,2,4]triazin-7-yl]methyl}piperazin-2-one hydrochloride hydrate (III) are obtained in the desired monohydrate [A] solid state form.

HPLC (Method 4):

purity: 99.9% (Rt=11.6 min.), relevant by-products: (VI) at RRT (relative retention time) of 0.16: n.d. (XVIII) at RRT 1.02 min: n.d.; (XV) at RRT 0.77:0.04%; (XVI) at RRT 0.98: n.d.

Assay for use: 98.2% (against external standard, based on monochloride, monohydrate)

Residual solvents (determined by GC method 1): 0.2% ethanol

Residual elements (determined by ICP-MS): 0.6 mg/kg palladium

Water content (Karl Fischer coloumetric): 3.8%

Ion chromatography: 6.8% chloride

### Example 10: Processes for Preparing Different Solid State Form Samples for Characterization on Laboratory Scale

Preparation of Monohydrate (A)

Crystalline form (III) as the monohydrate [A] of the compound of formula (I) was prepared as described above (according to Method 1, example 9)

Preparation of Dihydrate (B)

1 g Monohydrate form [A] of the compound of formula (II) (water content of 3.1%) was suspended in 10 mL methanol and stirred at 0° C. for five weeks. Subsequently the suspension was filtrated and the residue was stored at room temperature until the solvent had evaporated. It transformed into the Monohydrate in the course of two weeks.

Preparation of Trihydrate (C)

1 g Monohydrate form [A] of the compound of formula (II) (water content of 3.5%) was dissolved in 100 mL methanol under reflux. The solution was filtrated and stored in the refrigerator until the solvent had evaporated

Preparation of ¾-Hydrate (D)

3.5 g Monohydrate form [A] of the compound of formula (II) (water content of 3.1%) was suspended in 35 mL methanol and stirred at 0° C. for one week. Subsequently the suspension was filtrated and the residue was stored at room temperature until the solvent had evaporated

Preparation of Amorphous Material (E)

Monohydrate form [A] of the compound of formula (II) (water content of 3.1%) was spray dried with ethanol/water (1:1).

Physical characterization of hydrate forms of the compound of
formula (II) XRPD
Reflections (Peak maxima) [2 Theta]

| Monohydrate (A) | Dihydrate (B) | Trihydrate (C) | ¾-Hydrate (D) |
|---|---|---|---|
| 6.9 | 6.2 | 6.6 | 6.1 |
| 9.3 | 6.7 | 6.8 | 6.7 |
| 10.3 | 7.3 | 7.6 | 6.9 |
| 10.6 | 9.4 | 8.8 | 7.3 |
| 11.4 | 11.7 | 11.2 | 8.6 |
| 12.0 | 12.6 | 11.6 | 9.8 |
| 13.3 | 13.2 | 11.7 | 10.3 |
| 13.7 | 13.5 | 12.9 | 11.4 |
| 16.0 | 13.9 | 13.1 | 12.2 |
| 16.3 | 14.3 | 13.5 | 12.6 |
| 17.7 | 14.5 | 14.1 | 13.1 |
| 18.0 | 15.0 | 14.3 | 13.4 |
| 18.5 | 15.4 | 14.6 | 13.6 |
| 18.8 | 16.2 | 14.9 | 14.0 |
| 19.4 | 16.3 | 15.2 | 14.7 |
| 20.0 | 16.4 | 15.3 | 15.1 |
| 20.7 | 17.0 | 15.9 | 15.9 |
| 21.2 | 17.9 | 17.1 | 16.4 |
| 21.5 | 18.4 | 17.6 | 16.8 |
| 22.0 | 18.7 | 18.2 | 17.0 |
| 22.3 | 19.0 | 18.7 | 17.4 |
| 22.5 | 19.4 | 19.1 | 17.9 |
| 22.9 | 20.3 | 19.6 | 18.3 |
| 23.3 | 21.0 | 20.0 | 18.8 |
| 23.7 | 21.4 | 20.3 | 19.1 |
| 24.1 | 21.5 | 21.3 | 19.5 |
| 24.6 | 21.9 | 21.7 | 19.9 |
| 25.1 | 22.2 | 22.2 | 20.3 |
| 26.0 | 22.5 | 22.5 | 21.2 |
| 26.4 | 23.1 | 23.3 | 22.4 |
| 26.5 | 23.3 | 23.6 | 22.7 |
| 26.7 | 23.5 | 24.4 | 23.8 |
| 26.8 | 24.1 | 24.6 | 24.1 |
| 27.0 | 24.3 | 24.9 | 24.3 |
| 27.7 | 25.6 | 25.7 | 24.5 |
| 28.3 | 25.9 | 25.9 | 25.2 |
| 28.5 | 26.1 | 26.2 | 25.5 |
| 28.9 | 26.7 | 26.5 | 25.8 |
| 29.8 | 27.0 | 26.5 | 26.1 |
| 30.1 | 27.1 | 26.9 | 26.3 |
| 30.1 | 27.2 | 27.2 | 26.7 |
| 30.5 | 27.6 | 27.8 | 27.0 |
| 31.2 | 28.3 | 28.0 | 27.3 |
| 31.7 | 28.7 | 28.5 | 27.9 |
| 32.2 | 29.1 | 29.1 | 28.2 |
| 33.2 | 29.4 | 29.5 | 28.8 |
| 33.6 | 30.0 | 29.8 | 29.4 |
| 33.8 | 30.6 | 30.0 | 30.4 |
| 34.2 | 30.7 | 30.6 | 30.7 |
| 34.7 | 31.4 | 30.9 | 31.4 |
| 35.4 | 31.9 | 31.5 | 31.8 |
| 36.4 | 32.3 | 32.1 | 32.4 |
| 37.5 | 32.5 | 32.7 | 34.6 |
| | 32.8 | 33.2 | 35.3 |
| | 33.4 | 34.8 | 36.6 |
| | 34.4 | 35.9 | |
| | 35.0 | 36.9 | |
| | 35.8 | | |
| | 36.1 | | |
| | 37.1 | | |
| | 38.1 | | |
| | 38.5 | | |

Additional Data on Preparation and Characterization of Hydrate Forms of (II)

Preparation of hydrate forms of 4-{54-Amino-6-(methoxymethyl)-5-(7-methoxy-5-methyl-1-benzothiophen-2-yl)pyrrolo[2,1-f[1,2,4]triazin-7-yl]methyl}piperazin-2-one-mono-hydrochloride compound of formula (o)

Suspensions of monohydrate (A) were prepared in various solvents and stirred at 0° C. and 25° C. The residues were filtered off and dried at room temperature and ambient humidity. Table 1 summarizes the results. Reproducible preparation was not possible for all solid state forms.

TABLE 1
Slurry experiments

| | | Stirring | |
|---|---|---|---|
| | Temperature | time | Modification |
| Solvent | [° C.] | [weeks] | residue |
| Isopropanol | 0 | 1 | Monohydrate (A) |
| Methanol | 0 | 2 | ¾-Hydrate (D) |
| Methanol | 0 | 5 | Dihydrate (B) |
| Toluene | 25 | 4 | Monohydrate (A) |
| Tetrahydrofurane/water (1:1) | 25 | 4 | Monohydrate (A) |
| Acetonitrile/water (1:1) | 25 | 1 | Monohydrate (A) |
| Isopropanol | 25 | 4 | Monohydrate (A) |
| Ethanol | 25 | 4 | Monohydrate (A) |
| Ethanol/water (1:1) | 25 | 1 | Monohydrate (A) |
| Methanol | 25 | 1 | Monohydrate (A) |
| 1,4-Dioxane | 25 | 1 | Monohydrate (A) |

### Properties of hydrate forms of 4-{[4-Amino-6-(methoxymethyl)-5-(7-methoxy-5-methyl-1-benzothiophen-2-yl)pyrrolo[2,1-f] [1,2,4]triazin-7-yl]methyl}piperazin-2-one-mono-hydrochloride compound of formula (II)

Storage

The Monohydrate, ¾-Hydrate, the Trihydrate and the Amorphous form were stored in closed vessels at 25° C. and 50° C. (humidity not set). The results are summarized in Table 2.

TABLE 2
Storage at 25° C. and 50° C.

| | | Modification/after storage | |
|---|---|---|---|
| | Time | in closed vessels at | |
| Modification | [months] | 25° C. | 50° C. |
| Monohydrate | 12 | Monohydrate | Monohydrate |
| ¾-Hydrate | 12 | ¾-Hydrate | ¾-Hydrate |
| Trihydrate | 12 | Trihydrate | Amorphous + |
| | | | Trihydrate |
| Amorphous form | 6 | Amorphous form | Amorphous form |

Moisture Sorption

The Monohydrate, ¾-Hydrate, the Trihydrate and the Amorphous form were stored at various humidities. Their moisture content was then determined by thermogravimetric analysis. The results are summarized in Table 3.

TABLE 3

| | | Mass loss [%] and modification after | | |
|---|---|---|---|---|
| | Time | storage at 25° C. and | | |
| Modification | months | 15% r. h. | 85% r. h. | 97% r. h. |
| Monohydrate | 12 | 3.7 | 3.7 | 3.7 |
| | | Monohydrate | Monohydrate | Monohydrate |
| ¾-Hydrate | 12 | 2.6 | 4.1 | 7.9 |
| | | ¾-Hydrate | Monohydrate* | Monohydrate* |
| Trihydrate | 12 | — | 3.3 | 6.0 |
| | | | Monohydrate* | Monohydrate* |
| Amorphous form | 6 | — | 4.3 | 5.4 |
| | | | Monohydrate* | Monohydrate* |

*Transition into the Monohydrate already took place during one-month-storage.

Pharmaceutical Composition Containing Compound (III) (600 mg)

TABLE 4
Composition of 200 mg (API) tablets

| Composition | Amount [mg] |
|---|---|
| Drug substance | |
| Compound (III) ^{a} | 223.4 |
| Excipients | |
| Tablet core | |
| Cellulose microcrystalline | 139.1 |
| Copovidone | 12.2 |
| Crospovidone | 51.0 |
| Lactose monohydrate | 244.1 |
| Magnesium stearate | 6.8 |
| Purified water ^{b} | q.s. ^{b} |
| Silicia, colloidal, anhydrous | 3.4 |
| Weight (uncoated tablet) | 680.0 |
| Film-coating | |
| Ferric oxide red ^{c} | 1.85 |
| Hypromellose 15 cP ^{c} | 7.92 |
| Macrogol ^{c} | 2.64 |
| Titanium dioxide ^{c} | 0.79 |
| Purified water ^{b} | q.s. ^{b} |
| Weight (film-coating) ^{d} | 13.2 |
| Weight (coated tablet) | 693.2 |

^{a} (III) is the hydrochloride monohydrate of the free base (I), the dose strength 200 mg is corresponding to the amount of the free base. The drug substance is used in micronized form
^{b} q.s. = quantum sufficit (a sufficient quantity). Purified water is used as processing agent and quantitatively removed by the manufacturing process, except for low level of residual moisture. It does not contribute to the sum of mass of the drug product
^{c} Excipients may be used as a ready-to-use mixture
^{d} Theoretical amount

Description of the Tablet Manufacturing Process

Dry Blending

Cellulose microcrystalline, compound (III), lactose monohydrate and crospovidone were weighted in the proportions according to Table 1 into a suitable container and blended.

Wet Granulation

After blending the mixture was granulated with the binder solution (copovidone in purified water) in a high shear granulator. After granulation, the granulate was wet sieved.

Drying and Dry Milling

The sieved granules were transferred to the fluid bed dryer and dried until a LOD of 3-4% is reached.

After drying the granules were passed through a 0.9 mm sieve.

Final Blending

Cellulose microcrystalline, crospovidone and silica, colloidal, anhydrous were added to the final blend and mixed in the bin blender.

At last magnesium stearate was added to final blend and mixed.

Tablet Compression

The mixture was compressed on a rotary press into tablets with weight of 680.0 mg.

Coating

The tablets were coated into the drum coater with the coating suspension (hypromellose, macrogol, ferric oxide red, titanium dioxide in purified water).

Properties of Pharmaceutical Compositions Containing Compound (III)

Methods:

Tablets were assessed according to the following methods:

| | |
|---|---|
| Appearance | Appearance of tablets were assessed on a white |
| | background at daylight. |
| | Length and width were measured. |

| Identity | Identity was analyzed within the determination of | |
|---|---|---|
| (retention time) | assay. The retention time of compound I peak in the | |
| | sample must comply with the retention time of the | |
| | reference standard in reference assay within a range | |
| | of ± 5.0% | |
| | Solution | Inj. |
| Sequence for | Blank | 1 |
| determination of | Reference assay | 1 |
| identity | Sample | 1 |

| | |
|---|---|
| Identity (UV-spectrum) | The identity was analyzed with the determination |
| | of assay. The spectrum of the major peak in the |
| | chromatogram of the test solution should |
| | correspond to that of the major peak in the |
| | chromatogram of the compound I reference |
| | standard. |

Content Uniformity

| | |
|---|---|
| HPLC conditions | |
| Apparatus | Agilent HPLC 1100 or equivalent |
| Column | Agilent Zorbax Eclipse Plus C18 · 3.5 μm, |
| | 100 mm · 4.6 mm |
| Injection volume | 2 μL |
| | Injection with needle wash |
| Autosampler temperature | room temperature |
| Run time | 8 min |
| Wavelength | 226 nm |
| Eluent | 60% phosphate buffer |
| | 40% acetonitrile |
| Flow | 0.5 mL/min |
| Column temperature | 45° C. |
| Vials | amber glass |
| Reagents and solutions | |
| Phosphate buffer | Weigh 1.15 g ammonium dihydrogen phosphate into a |
| | 1000 mL volumetric flask. Add 0.68 mL phosphoric |
| | acid (ultra-pure, conc.) and fill up to mark with water. |
| | Check pH and adjust to 2.40-2.50 with phosphoric |
| | acid or prepare new. Preparation of differing volumes |
| | is also possible. Amounts of buffering agent, |
| | phosphoric acid, and water then need to be adjusted |
| | appropriately. |
| Diluent | Mix water and acetonitrile in exactly equal volume shares. |
| Blank solution (blank) | Filter diluent through a 0.2 μm PTFE filter and |
| | discard the first few milliliters. |
| SST | Use reference solution. |
| Reference solution (reference) | Prepare solution of coumpound III reference |
| | substance in diluent using amber glass volumetric |
| | flask. Final concentration of compound I should be |
| | 0.1 mg/mL. Sonicate until dissolved. |
| | Stable for 14 days stored at room temperature and daylight. |
| Sample solution 1 | Analysis will be performed with 10 single units. |
| | Weigh and transfer one tablet into a 100 mL amber |
| | volumetric flask. Add 70 mL diluent and sonicate to |
| | suspend. Cool down to room temperature, fill up to |
| | mark with diluent and mix well. Filter through a |
| | 0.2 μm PTFE filter and discard the first few |
| | milliliters. |
| | Preparation time should be less than 120 minutes |
| Sample solution 2 (sample) | Dilute sample solution 1 to theoretical concentration |
| | of 0.1 mg/mL of compound I with diluent. Use amber |
| | flask. Stable for 7 days stored at room temperature |
| | and daylight. |

Sequence

| Solution | Inj. |
|---|---|
| Blank | 1 |
| SST | 6 |
| Reference | 2 |
| Sample 1-10 | 1 |
| Reference | 2 |
| Calculation: | |
| cc. [%] = | $\frac{weight reference [ mg ] \cdot P [ \% ] \cdot 100 \%}{100 \% sample dosage per tablet [ 200 mg ]} \cdot {f}_{d}$ |
| | P = Purity of reference substance |
| | f_{d} = dilution factor |
| sample [%] = | $\frac{area sample \cdot cc .}{area reference}$ |
| | cc. = Calibration concentration |
| Acceptance criteria: | |
| Blank | No interfering peaks at the retention time of |
| | compound I. |
| SST | Symmetry factor (Ph. Eur.) is between 0.8 and 1.5 |
| | RSD peak area (n = 6) ≤ 2.0% |
| Reference (within bracketing) | RSD peak area (n = 4) ≤ 2.0% |
| Acceptance value (USP <905> / | The uniformity of dosage units of the tablets must |
| Ph. Eur. 2.9.40) | meet the requirement of the cited quality |
| | specification. If the acceptance value (AV) of the |
| | 10 tablets is > 15.0%, test additional 20 tablets and |
| | calculate the acceptance value of 30 tablets. |
| Reporting: | AV |
| | If stage 2 than also: Min, Max |

Degradation products

| | |
|---|---|
| Integration | Impurities are analyzed within the determination of |
| | assay. Degradation products can be quantified in the |
| | sample solutions for up to 7 days |
| | SST1 has to be reported with an own process method |
| | for calculation to calculate the resolution (compound I |
| | to RRT 0.96 and RRT 1.08) with the integration |
| | parameter valley to valley. |
| | The peak RRT 0.96 (NK5) to compound I in all other |
| | chromatograms has to be integrated by drop down and |
| | the peak with a RRT 1.08 (NK11) to compound I has |
| | to be integrated by tangential skim. |

Dissolution

| | |
|---|---|
| Dissolution conditions | |
| Apparatus | Paddle |
| Dissolution medium | 900 mL hydrochloric acid pH 2.0 |
| | For 6 L: Dilute 6 mL hydrochloric acid (32%) with |
| | 5994 mL purified water. Adjust to pH 2 if necessary. |
| Stirring speed | 50 rpm ± 2 rpm |
| Temperature | 37.0° C. ± 0.5° C. |
| Sample withdrawal | 10 mL each after 5, 10, 15, 30, and 45 min (infinity) |
| Infinity test | after 30 min withdrawal adjust the stirring speed to 200 rpm |
| HPLC conditions | |
| Apparatus | Agilent HPLC 1100 or equivalent |
| Column | Agilent Zorbax Eclipse Plus C18 · 3.5 μm, 100 mm · 4.6 mm |
| Injection volume | 2 μL |
| | Injection with needle wash (1x) |
| Autosampler temperature | room temperature |
| Run time | 8 min |
| Wavelength | 226 nm |
| Eluent | 60% phosphate buffer pH 2.4 |
| | 40% acetonitrile |
| Flow | 0.5 mL/min |
| Column temperature | 45° C. |
| Vials | amber glass |
| Reagents and solutions | |
| Phosphate buffer pH 2.4 | For 1 L: Weigh 1.15 g ammonium dihydrogen |
| | phosphate into a 1000 mL volumetric flask. Add |
| | 0.68 mL phosphoric acid (ultra-pure, conc.) and fill up |
| | to mark with water. Check pH and adjust to 2.4 |
| | (2.35-2.50) with phosphoric acid or prepare new. |
| | For preparation of differing volumes, adjust amounts |
| | of buffering agent, phosphoric acid, and water |
| | appropriately. |
| Diluent | Mix water and acetonitrile in exactly equal volume shares. |
| Blank solution (blank) | Pipette 2.0 mL diluent into a 20 mL amber volumetric |
| | flask and fill up to mark with dissolution medium. |
| | Filter through a 0.45 μm RC filter and discard the first |
| | few milliliters. |
| SST | Use reference solution 2. |
| Reference solution 1 | Prepare solution of compound III reference substance |
| | in diluent using amber glass volumetric flask. Final |
| | concentration of compound I should be 2.20 mg/mL. |
| | Sonicate until dissolved. |
| | Stable for 14 days if stored in refrigerator at 2° C.-8° C. |
| Reference solution 2 (reference) | Dilute reference solution 1 to theoretical |
| | concentration of 0.22 mg/mL of compound I with |
| | dissolution medium. Use amber flask |
| | Stable for 14 days. Store in refrigerator at 2° C.-8° C. |
| Sample solution (sample) | Weigh 1 tablet and transfer it to one vessel. Repeat |
| | this procedure for 6 vessels. Withdraw a sample of |
| | 10 mL after the appropriate time point and filter |
| | through a 0.45 μm RC filter. Discard the first 6 mL |
| | and analyze. No medium is replaced. Use one syringe |
| | and one filter for each sampling time point. |
| | Stable for 3 days, stored at room temperature and daylight. |

Sequence

| Solution | Inj. |
|---|---|
| Blank | 1 |
| SST | 6 |
| Reference | 2 |
| Sample 1-30 | 1 |
| Reference | 2 |
| Calculation | |
| Reference: cc. [mg/mL] = | $\frac{weight reference [ mg ] \cdot P [ \% ]}{100 \%} \cdot {f}_{d}$ |
| | P = Purity of reference substance |
| | f_{d} = dilution factor |
| sample [mg/mL] = | $\frac{area sample \cdot cc .}{area reference}$ |
| | Calculation of concentration in % is automatically |
| | performed by the chromatography software. |
| | cc. = Calibration concentration |
| Acceptance criteria | |
| Blank | No interfering peaks at the retention time of compound I |
| SST | Symmetry factor (Ph. Eur.) is between 0.8 and 1.5 |
| | RSD area (n = 6) ≤ 2.0% |
| | (cf. Fehler! Verweisquelle konnte nicht gefunden werden.) |
| Reference (within bracketing) | RSD area (n = 4) ≤ 2.0% |
| | (cf. Fehler! Verweisquelle konnte nicht gefunden werden.) |
| Reporting (specified after 30 min) | Average, Min, Max, Evaluation |

TABLE 5
Coated tablet 200 mg, packaged in HDPE-bottles

| Test | Storage time | 25° C./ | 30° C./ | 40° C./ |
|---|---|---|---|---|
| Acceptance criterion | [months] | 60% RH | 75% RH | 75% RH |
| Crystalline form (III) | Initial | confirmed | — | confirmed |
| | 1 | confirmed | — | confirmed |
| | 3 | — | — | confirmed |
| | 6 | confirmed | — | confirmed |
| | 9 | — | — | — |
| | 12 | confirmed | — | — |
| | 18 | — | — | — |
| | 24 | confirmed | — | — |
| | 36 | confirmed | — | — |
| | 48 | confirmed | — | — |
| | 60 | | | |

Tablets with compound III were manufactured according to the method described above and an initial XRPD was recorded. The XRPD pattern remained unchanged at all storage condition investigated over a period of 48 months.

TABLE 6
Stability in brown glass bottle

| | Storage | | |
|---|---|---|---|
| | time | | |
| Test item | (months) | 25° C./60% r.h. | 40° C./75% r.h. |
| Material | initial | slightly grey-tinged solid | |
| and colour | 3 | slightly grey-tinged solid | slightly grey-tinged solid |
| | 6 | slightly grey-tinged solid | slightly grey-tinged solid |
| | 9 | slightly grey-tinged solid | n.t. |
| | 12 | slightly grey-tinged solid | n.t. |
| | 18 | slightly grey-tinged solid | n.t. |
| | 24 | slightly grey-tinged solid | n.t. |
| Water [%] | initial | 3.5 | |
| | 3 | 3.9 | 3.6 |
| | 6 | 3.5 | 3.4 |
| | 9 | 3.5 | n.t. |
| | 12 | 3.6 | n.t. |
| | 18 | 3.6 | n.t. |
| | 24 | 3.5 | n.t. |
| Ethanol | initial | 0.533 | |
| [%] | 3 | 0.460 | 0.406 |
| | 6 | 0.467 | 0.435 |
| | 9 | 0.444 | n.t. |
| | 12 | 0.441 | n.t. |
| | 18 | 0.434 | n.t. |
| | 24 | 0.415 | n.t. |
| Sum of | initial | 1.03 | |
| all organic | 3 | 1.08 | 1.17 |
| impurities | 6 | 1.05 | 1.00 |
| [%] | 9 | 1.14 | n.t. |
| | 12 | 1.22 | n.t. |
| | 18 | 1.17 | n.t. |
| | 24 | 1.14 | n.t. |
| Assay calc. | initial | 100.3 | |
| on dried | 3 | 99.8 | 98.4 |
| substance | 6 | 100.0 | 99.7 |
| [%] | 9 | 100.0 | n.t. |
| | 12 | 98.1 | n.t. |
| | 18 | 100.4 | n.t. |
| | 24 | 98.8 | n.t. |

TABLE 7
Stability of tablet
200 mg strength, HDPE-bottles, concomitant study

| Test | Storage time | 25° C./ | 30° C./ | 40° C./ |
|---|---|---|---|---|
| Acceptance criterion | [months] | 60% RH | 75% r.h. | 75% r.h. |
| Appearance | Initial | no change | no change | no change |
| (formulation, | 1 | no change | no change | no change |
| form, color) | | | | |
| dark red, oblong, | 3 | no change | no change | no change |
| coated tablet | 6 | no change | no change | no change |
| | 9 | no change | no change | — |
| | 12 | no change | no change | — |
| | 18 | no change | no change | — |
| | 24 | no change | no change | — |
| | 36 | no change | no change | — |
| | 48 | no change | no change | — |
| | 60 | | | |
| Dissolution | Initial | 81 | 81 | 81 |
| after 30 minutes | 1 | 82 | 81 | 80 |
| Mean % | 3 | 80 | 78 | 83 |
| | 6 | 78 | 76 | 74/97 ^{a, b} |
| Q = 75% | 9 ^{a} | 99 | 100 | — |
| | 12 | 95 | 97 | — |
| | 18 | 95 | 95 | — |
| | 24 | 102 | 100 | — |
| | 36 | 95 | 92 | — |
| | 48 | 96 | 96 | — |
| | 60 | | | |
| Water | Initial | 3.9 | 3.9 | 3.9 |
| [%] | 1 | 4.1 | 4.5 | 4.0 |
| | 3 | 3.9 | 4.0 | 4.2 |
| ≤8% | 6 | 4.5 | 4.9 | 4.8 |
| | 9 | 4.7 | 4.7 | — |
| | 12 | 4.6 | 4.3 | — |
| | 18 | 5.1 | 5.5 | — |
| | 24 | 4.2 | 4.5 | — |
| | 36 | 4.4 | 4.9 | — |
| | 48 | 4.7 | 5.3 | — |
| | 60 | | | |
| Degradation products | Initial | <0.05 | <0.05 | <0.05 |
| Any unspecified | 1 | <0.05 | <0.05 | <0.05 |
| degradation product | 3 | 0.06 | 0.06 | 0.06 |
| ≤0.6 % | 6 | 0.05 | 0.05 | 0.05 |
| | 9 | 0.05 | 0.05 | — |
| | 12 | 0.05 | 0.05 | — |
| | 18 | <0.05 | <0.05 | — |
| | 24 | <0.05 | 0.05 | — |
| | 36 | 0.05 | 0.06 | — |
| | 48 | 0.06 | 0.05 | — |
| | 60 ^{c} | | | |
| Sum of all | Initial | <0.05 | <0.05 | <0.05 |
| degradation products | 1 | <0.05 | <0.05 | <0.05 |
| | 3 | 0.06 | 0.06 | 0.11 |
| ≤5.0 % | 6 | 0.05 | 0.05 | 0.05 |
| | 9 | 0.05 | 0.05 | — |
| | 12 | 0.05 | 0.05 | — |
| | 18 | <0.05 | <0.05 | — |
| | 24 | <0.05 | 0.05 | — |
| | 36 | 0.05 | 0.06 | — |
| | 48 | 0.06 | 0.05 | — |
| | 60 ^{c} | | | |
| Assay | Initial | 203.8 | 203.8 | 203.8 |
| 190.0-210.0 | 1 | 208.3 | 206.2 | 205.4 |
| mg/tablet | 3 | 201.4 | 200.8 | 200.0 |
| | 6 | 201.1 | 204.9 | 201.6 |
| | 9 | 201.2 | 201.6 | — |
| | 12 | 200.3 | 202.9 | — |
| | 18 | 203.0 | 206.7 | — |
| | 24 | 202.9 | 200.3 | — |
| | 36 | 201.3 | 201.9 | — |
| | 48 | 198.2 | 205.8 | — |
| | 60 | | | |

^{a} Effective date of dissolution method change
^{b} Dissolution tested after 7 months
^{c} First application of updated evaluation method for degradation products

### FIGURES

FIG. 1: X-Ray powder diffractogram of the Monohydrate (A) according to Example 10.

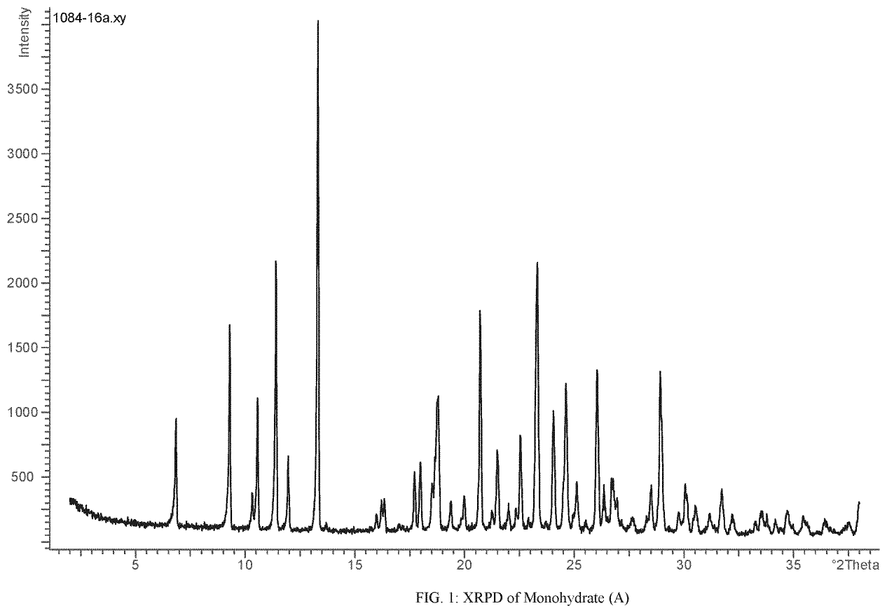

FIG. 2: X-Ray powder diffractogram of the Dihydrate (B) according to Example 10.

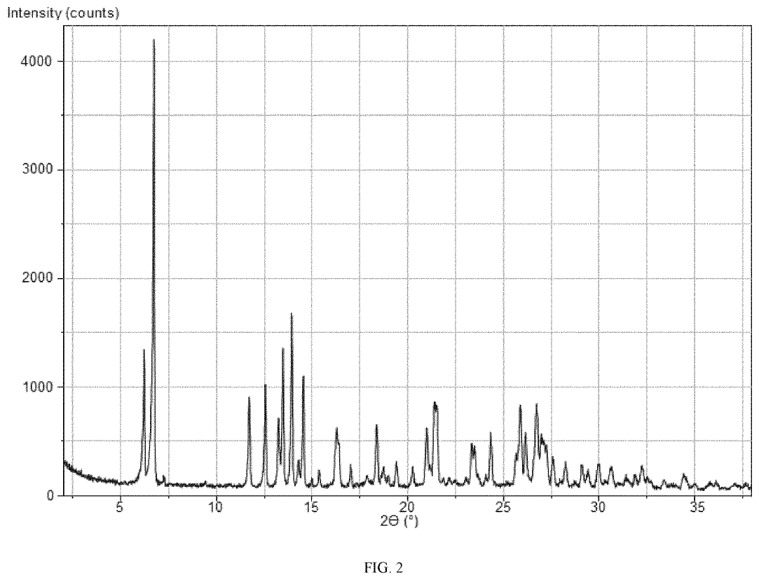

FIG. 3: X-Ray powder diffractogram of the Trihydrate (B) according to Example 10.

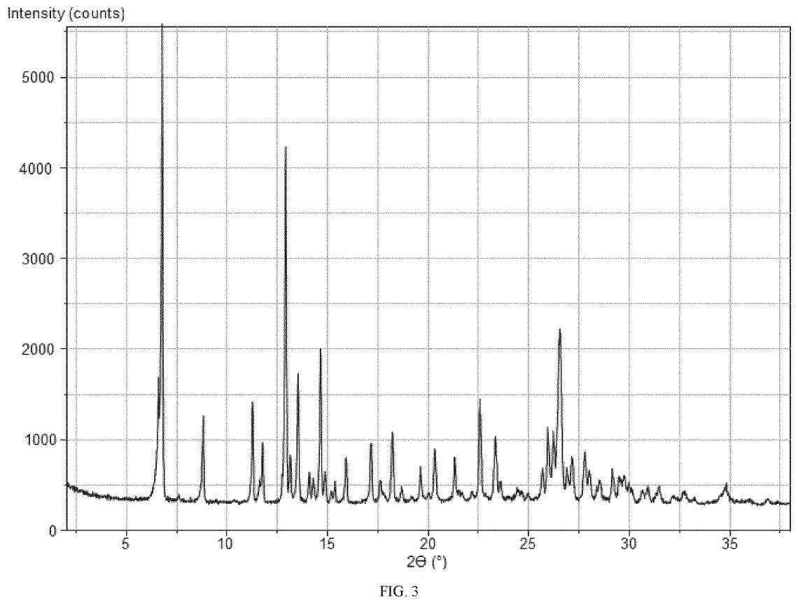

FIG. 4: X-Ray powder diffractogram of the ¾-hydrate (D) according to Example 10.

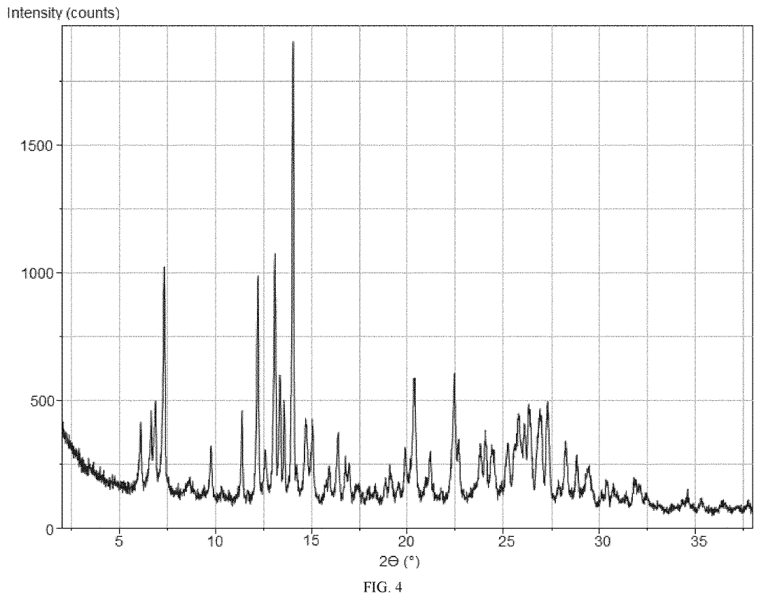

1: A compound of formula (III)

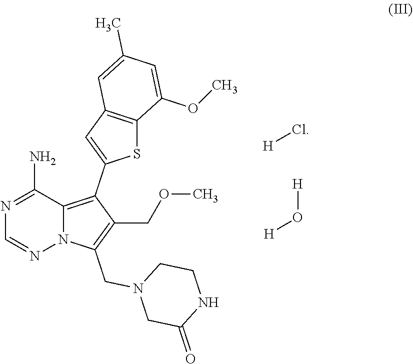 [SMILES: COCc1c(-c2cc3cc(C)cc(OC)c3s2)c2c(N)ncnn2c1CN1CCNC(=O)C1 ; Cl ; O]

2: The compound of claim 1, wherein the compound is in a form having a X-ray powder diffractogram measured at 25° C. and with Cu—K alpha 1 as radiation source displaying at least the following reflections, quoted as 2θ value ±0.2°: 9.3, 10.6, and 13.3.

3: The compound of claim 1, wherein the compound is in a form having a X-ray powder diffractogram measured at 25° C. and with Cu—K alpha 1 as radiation source displaying at least the following reflections, quoted as 2θ value ±0.2°: 9.3, 10.6, 13.3, 20.7, and 23.3.

4: The compound of claim 1, wherein the compound is in a form having a X-ray powder diffractogram measured at 25° C. and with Cu—K alpha 1 as radiation source displaying at least the following reflections, quoted as 2θ value ±0.2°: 9.3, 10.6, 11.4, 13.3, 20.7, 23.3, and 26.0.

5: The compound of claim 1, wherein the compound is in a form having a X-ray powder diffractogram measured at 25° C. and with Cu—K alpha 1 as radiation source displaying at least the following reflections, quoted as 2θ value ±0.2°: 6.8, 9.3, 10.6, 11.4, 13.3, 20.7, 23.3, 24.6, 26.0, and 27.6.

6: A pharmaceutical composition comprising a pseudopolymorphic form of the compound of formula (III) according to claim 1 and optionally further pharmaceutically acceptable excipients.

7: The pharmaceutical composition of claim 6, wherein the pseudopolymorphic form of the compound of formula (III) is a crystalline form and wherein the composition comprises mainly the crystalline form of the compound of formula (III) and no significant fractions of another form of the compound of the formula (I) 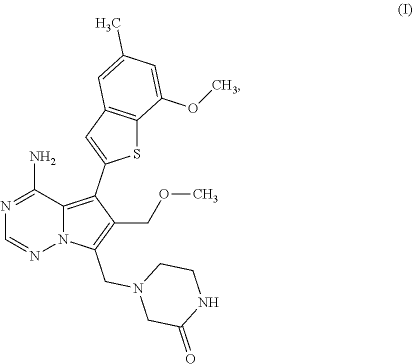 [SMILES: COCc1c(-c2cc3cc(C)cc(OC)c3s2)c2c(N)ncnn2c1CN1CCNC(=O)C1]
and optionally further pharmaceutically acceptable excipients.

8: (canceled)

9: A method for treating or preventing proliferative disorders comprising administering to a mammal in need thereof a therapeutically effective amount of a pharmaceutical composition according to claim 6.

10: (canceled)

11: (canceled)

12: A method of treating or preventing proliferative disorders in a mammal, comprising administering to a mammal in need thereof a therapeutically effective amount of the compound according to claim 1.

13: A process for producing the compound according to claim 1, comprising dissolving or suspending the compound of formula (I) in an inert solvent and adding an acid or acid precursor.

14: The process of claim 13, comprising dissolving or suspending compound of formula (I) in THF or EtOH and water, and adding HCl.

15: The process of claim 14, further comprising reacting a compound of formula (VII) 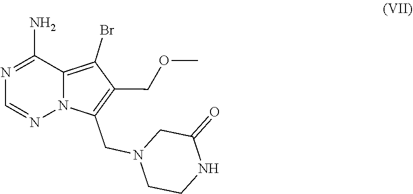 [SMILES: COCc1c(Br)c2c(N)ncnn2c1CN1CCNC(=O)C1]
in the presence of THF and water with K₂CO₃ and a palladium catalyst with a compound of formula (VIII) 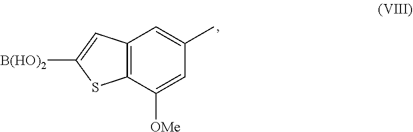
thereby producing the compound of formula (I).

16: The process of claim 15, further comprising reacting a compound of formula (V) 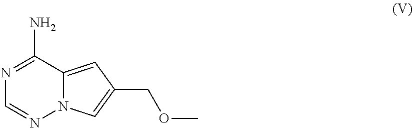
with a compound of formula (XIII) 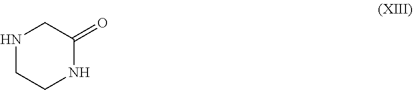
with paraformaldehyde in the presence of an acid thereby producing a compound of formula (VI), 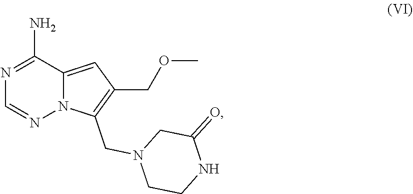 [SMILES: COCc1cc2c(N)ncnn2c1CN1CCNC(=O)C1]
and then reacting the compound of formula (VI) with a bromination agent, thereby producing the compound of formula (VII).

17: The process of claim 16, further comprising reacting a compound of formula (IV) 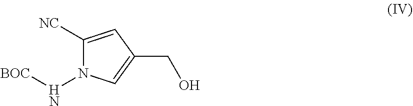 [SMILES: CC(C)(C)OC(=O)Nn1cc(CO)cc1C#N]
with an acid thereby producing a compound of formula (XXII), 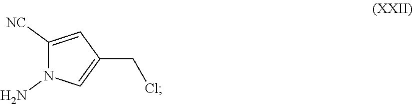
reacting the compound of formula (XXII) with methanol in the presence of a base, thereby producing a compound of formula (XXIII), 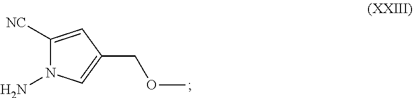
and then reacting the compound of formula (XXIII) with formamidine acetate and a base, thereby producing the compound of formula (V).

18: The process of claim 17, further comprising reacting a compound of formula (X) 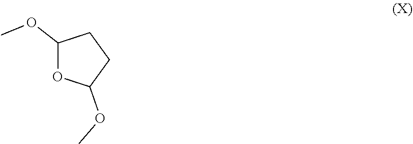
with Boc-NH—NH₂ to form a compound of formula (XI) 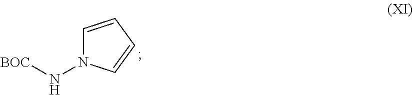 [SMILES: CC(C)(C)OC(=O)Nn1cccc1]
reacting the compound of formula (XI) with CISO₂NCO to form a compound of formula (XII) 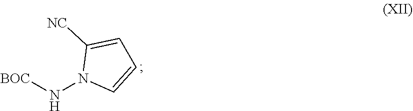 [SMILES: CC(C)(C)OC(=O)Nn1cccc1C#N]
reacting the compound of formula (XII) with N-bromosuccinimide to form a compound of formula (IX) 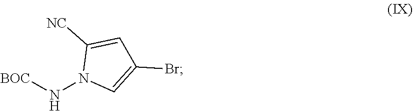 [SMILES: CC(C)(C)OC(=O)Nn1cc(Br)cc1C#N]
and
reacting the compound of formula (IX) with metal organic reagents in addition to paraformaldehyde, thereby producing the compound of formula (IV).

19: The method of claim 9, wherein the proliferative disorders are cancer or tumor diseases.

20: The method of claim 12, wherein the proliferative disorders are cancer or tumor diseases.

21: The method of claim 18, wherein the metal organic reagents are methyl magnesium bromide and butyl lithium.
